(19) United States
(12) Patent Application Publication
(10) Pub. No.: US 2023/0407009 A1
(43) Pub. Date: Dec. 21, 2023

(54) POLYMER, ORGANIC SEMICONDUCTOR MATERIAL, STRETCHABLE POLYMER THIN FILM, AND ELECTRONIC DEVICE

(71) Applicants: Samsung Electronics Co., Ltd., Suwon-si (KR); THE BOARD OF TRUSTEES OF THE LELAND STANFORD JUNIOR UNIVERSITY, Stanford, CA (US)

(72) Inventors: Sangah GAM, Suwon-si (KR); Deyu LIU, Stanford, CA (US); Zhenan BAO, Stanford, CA (US); Youngjun YUN, Stanford, CA (US)

(73) Assignees: Samsung Electronics Co., Ltd., Suwon-si (KR); THE BOARD OF TRUSTEES OF THE LELAND STANFORD JUNIOR UNIVERSITY, Stanford, CA (US)

(21) Appl. No.: 18/337,581

(22) Filed: Jun. 20, 2023

Related U.S. Application Data
(60) Provisional application No. 63/353,669, filed on Jun. 20, 2022.

(51) Int. Cl. C08G 75/06; H10K 85/10

(52) U.S. Cl. CPC C08G 75/06; H10K 85/113

(57) ABSTRACT

Disclosed are a polymer including a first structural unit represented by Chemical Formula 1 and a second structural unit represented by Chemical Formula 2, an organic semiconductor material, and a stretchable polymer thin film and electronic device including the same.

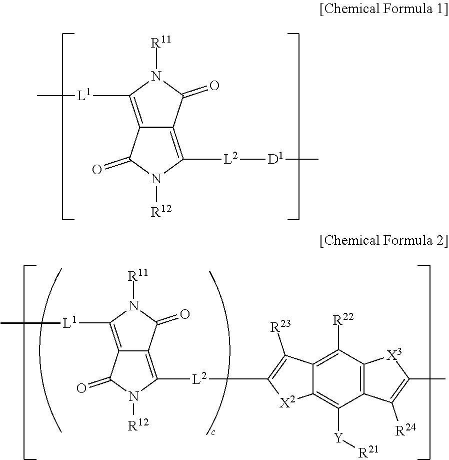

Chemical Formulas 1 and 2 are described in the detailed description.

### CROSS-REFERENCE TO RELATED APPLICATION

This application claims priority under 35 U.S.C. § 119 to U.S. Patent Application No. 63/353,669 filed in the United States Patent and Trademark Office on Jun. 20, 2022, the entire contents of which are incorporated herein by reference.

### BACKGROUND

### 1. Field

A polymer, an organic semiconductor material, a stretchable polymer thin film, and an electronic device are disclosed.

### 2. Description of the Related Art

In recent years, research on a stretchable display device and/or a bio-attachable device such as a smart skin device, a soft robot, and a biomedical device has been conducted. In addition to electrical characteristics, these devices should have stretchability in an arbitrary direction according to external movements, and at the same time should be able to maintain their original performance after being restored, and thus a new material suitable for this may be required.

### SUMMARY

Some example embodiments provide a polymer with excellent electrical properties such as charge mobility and stretchability.

Some example embodiments provide an organic semiconductor material including the polymer.

Some example embodiments provide a stretchable polymer thin film including the polymer or organic semiconductor material.

Some example embodiments provide an electronic device including the polymer, the organic semiconductor material, or the stretchable polymer thin film.

According to some example embodiments, a polymer including a first structural unit represented by Chemical Formula 1 and a second structural unit represented by Chemical Formula 2 is provided.

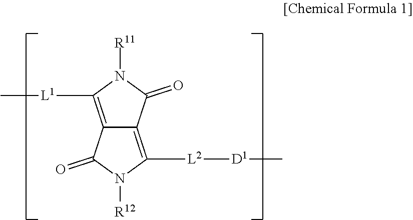

In Chemical Formula 1,
R¹¹ and R¹² may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, —COR^{a}, OC(═O)R^{b}, —C(═O)OR^{c}, —OC(═O)OR^{d}, a halogen, a cyano group, or any combination thereof, wherein R^{a} to R^{d} may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, a cyano group, or any combination thereof,
L¹ and L² may each independently be a single bond, a substituted or unsubstituted C6 to C30 arylene group, a substituted or unsubstituted divalent C3 to C30 heterocyclic group, a fused ring thereof, or any combination thereof, and
D¹ may be a substituted or unsubstituted C6 to C30 arylene group; a substituted or unsubstituted divalent C3 to C30 heterocyclic group including at least one of N, O, S, Se, Te, and Si; a fused ring thereof; or any combination thereof.

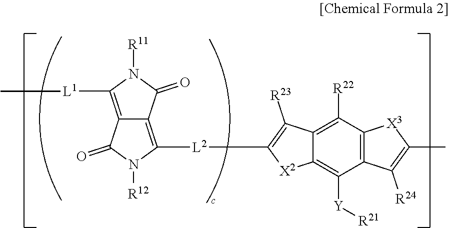

### in Chemical Formula 2,

R¹¹ and R¹² may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, —COR^{a}, —OC(═O)R^{b}, —C(═O)OR^{b}, —OC(═O)OR^{d}, a halogen, a cyano group, or any combination thereof, wherein R^{a} to R^{d} may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, a cyano group, or any combination thereof,
L¹ and L² may each independently be a single bond, a substituted or unsubstituted C6 to C30 arylene group, a substituted or unsubstituted divalent C3 to C30 heterocyclic group, a fused ring thereof, or any combination thereof,
c may be 0 or 1,
Y may be O, C(═O), —OC(═O), —C(═O)O, or —OC(═O)O,
R²¹ may be a substituted or unsubstituted C1 to C30 straight chain or branched chain alkyl group, a C3 to C30 linear or branched alkyl group in which at least one of non-adjacent methylene groups is replaced by oxygen (O), a substituted or unsubstituted C3 to C30 cycloalkyl group, or a substituted or unsubstituted C3 to C30 heterocycloalkyl group,
R²² may be a substituted or unsubstituted C6 to C30 aryl group, a substituted or unsubstituted C7 to C30 aralkyl group, a substituted or unsubstituted C2 to C30 heteroaryl group, a substituted or unsubstituted C3 to C30 cycloalkyl group, or a substituted or unsubstituted C3 to C30 heterocycloalkyl group,
R²³ and R²⁴ may each independently be hydrogen, a substituted or unsubstituted C1 to C30 linear or branched alkyl group, a substituted or unsubstituted C1 to C30 linear or branched alkoxy group, —COR^{a}, —OC(═O)R^{b}, —C(═O)OR^{c}, —OC(═O)OR^{d}, a halogen, or a cyano group, wherein R a to R d may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, or a cyano group, and
X² and X³ are each independently O, S, Se, or Te.

In some embodiments, in Chemical Formula 1, D¹ may be at least one substituted or unsubstituted phenylene group; at least one substituted or unsubstituted naphthylene group; at least one substituted or unsubstituted anthracenylene group; at least one substituted or unsubstituted phenanthrenylene group; at least one substituted or unsubstituted pentagonal ring group including at least one selected from N, O, S, Se, Te, and Si; a fused ring of two or more of the above substituted or unsubstituted pentagonal ring groups; a fused ring of at least one substituted or unsubstituted pentagonal ring group and at least one substituted or unsubstituted phenylene group; a fused ring of at least one substituted or unsubstituted pentagonal ring group and at least one substituted or unsubstituted naphthylene group; a fused ring of at least one substituted or unsubstituted pentagonal ring group and at least one substituted or unsubstituted anthracenylene group; a fused ring of at least one substituted or unsubstituted pentagonal ring group and at least one substituted or unsubstituted phenanthrenylene group; or any combination thereof.

In some embodiments, D 1 in Chemical Formula 1 may be, for example, one of electron donating moieties listed in Group 1.

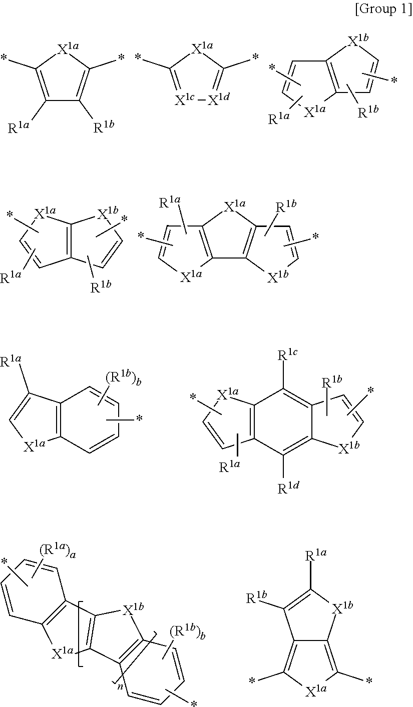

In Group 1,
X^{1a} and X^{1b} may each independently be O, S, Se, or Te,
X^{1c} and X^{1d} may each independently be N, CR^{x}, or SiR^{y},
X^{1e} is O, S, Se, Te, NR^{v}, CR^{w}R^{x}, or SiR^{y}R^{z},
R^{1a}, R^{1b}, R^{1c}, R^{v}, R^{w}, R^{x}, R^{y}, and R^{z} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
a and b are each independently an integer ranging from 1 to 4,
n is 0, 1, or 2, and
* is a linking point.

In some embodiments, in Chemical Formula 1, L¹ and L² may each independently be a single bond; one of electron donating moieties listed in Group 1; a substituted or unsubstituted pyridine; a substituted or unsubstituted pyrimidine; a fused ring thereof; or any combination thereof.

In some embodiments, in Chemical Formula 1, L¹ and L² may each independently be a divalent linking group including a single bond; at least one substituted or unsubstituted furan; at least one substituted or unsubstituted thiophene; at least one substituted or unsubstituted selenophene; at least one substituted or unsubstituted tellurophene; at least one substituted or unsubstituted pyrrole; at least one substituted or unsubstituted benzene; at least one substituted or unsubstituted pyridine; at least one substituted or unsubstituted pyrimidine; a fused ring of two or more selected from the foregoing groups; or any combination thereof.

In some embodiments, in Chemical Formula 1, R¹¹ and R¹² may each independently be a substituted or unsubstituted C6 to C30 linear alkyl group or a substituted or unsubstituted C6 to C30 branched alkyl group.

In some embodiments, in Chemical Formula 2, R²² may be one of moieties represented by Group 2.

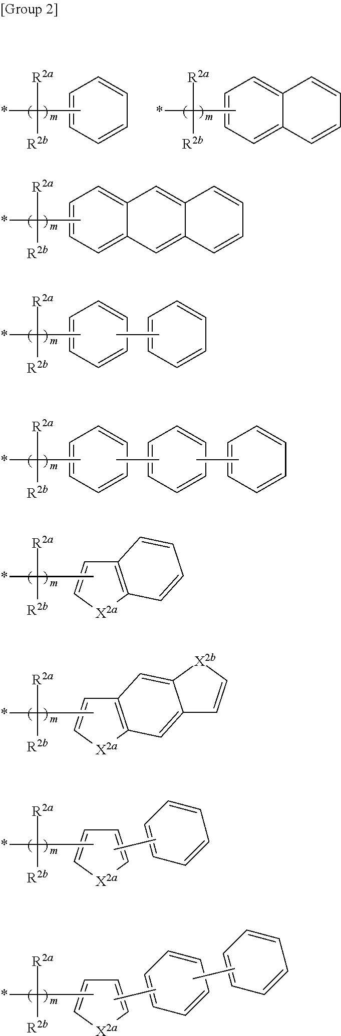

In Group 2,
X^{2a} and X^{2b} may each independently be O, S, Se, or Te,
R^{2a} and R^{2b} may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
m is an integer ranging from 0 to 10, and
* is a linking point.

In some embodiments, in Group 2, a hydrogen atom of the benzene ring may be substituted or unsubstituted with a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group.

In some embodiments, in Group 2, at least one (CH₂) group in the benzene ring may be replaced by nitrogen (N).

In some embodiments, in —(CR^{2a}R^{2b})ₘ— of Group 2, when m is 2 or more and 10 or less, —CR^{2a}R^{2b}— that are not adjacent to each other may be replaced by —O—, —S—, —C(═O)—, —OC(═O)—, —C(═O)O—, —OC(═O)O—, —S(═O)—, —S(═O)₂—, or any combination thereof.

In some embodiments, in Chemical Formula 2, R^{21 ,} R^{23 ,} and R²⁴ independently may be a substituted or unsubstituted C6 to C30 linear alkyl group or a substituted or unsubstituted C6 to C30 branched alkyl group.

In some embodiments, in the polymer, an aspect ratio (Z/X) obtained by dividing the shortest axis length (Z) by the longest axis (X) length of a compound including one first structural unit and one second structural unit may be less than or equal to about 1.4.

In some embodiments, the first structural unit and the second structural unit may be included in a molar ratio of about 1:9 to about 9:1.

According to some example embodiments, an organic semiconductor material including the polymer may be provided.

According to some example embodiments, a stretchable polymer thin film including the polymer or organic semiconductor material may be provided.

In some embodiments, the stretchable polymer thin film may further include an elastomer.

In some embodiments, when the stretchable polymer thin film is stretched by about 30%, a change in charge mobility may be less than or equal to about 10%.

According to some example embodiments, an electronic device including the stretchable polymer thin film may be provided.

In some embodiments, the electronic device may include an organic diode, an organic thin film transistor, an organic solar cell, or an attachable device.

The polymer may simultaneously satisfy electrical properties such as charge mobility and stretchability, and thus may be effectively applied to stretchable electronic devices.

### BRIEF DESCRIPTION OF THE DRAWINGS

FIGS. 1 to 3 are cross-sectional views each showing a thin film transistor according to some example embodiments.

FIGS. 4 to 8 are optical micrographs showing presence or absence of cracks according to the elongation rate of the polymer thin films according to Examples 1 to 4 and Comparative Example 1, respectively.

FIG. 9 is a circuit diagram of an inverter according to an example embodiment.

FIG. 10 is a block diagram of an electronic device according to an example embodiment.

### DETAILED DESCRIPTION

Hereinafter, some example embodiments are described in detail so that those skilled in the art can easily implement them. However, the actual applied structure may be implemented in various different forms and is not limited to the implementations described herein.

In the drawings, the thickness of layers, films, panels, regions, etc., are exaggerated for clarity.

It will be understood that when an element or layer is referred to as being “on,” “connected to,” “coupled to,” or “covering” another element or layer, it may be directly on, connected to, coupled to, or covering the other element or layer, or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It will be understood that, although the terms first, second, third, etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the example embodiments.

Spatially relative terms, e.g., “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various embodiments only and is not intended to be limiting of the example embodiments. As used herein, the singular forms “a,” “an,” and “the” are intended to include the plural forms as well, unless the context clearly indicates otherwise. It will be further understood that the terms “comprises,” “comprising,” “includes,” and/or “including,” if used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art. It will be further understood that terms, including those defined in commonly used dictionaries, should be interpreted as having a meaning that is consistent with their meaning in the context of the relevant art and will not be interpreted in an idealized or overly formal sense unless expressly so defined herein.

In the drawings, parts having no relationship with the description are omitted for clarity of the embodiments, and the same or similar constituent elements are indicated by the same reference numerals throughout the disclosure.

In the present disclosure, the term “or” is not to be construed as an exclusive meaning, and for example, “A or B” is construed to include A, B, A+B, and/or the like.

As used herein, “at least one of A, B, or C,” “one of A, B, C, or any combination thereof” and “one of A, B, C, and any combination thereof” refer to each constituent element, and any combination thereof (e.g., A; B; C; A and B; A and C; B and C; or A, B, and C).

Hereinafter, the terms “lower portion” and “upper portion” are for convenience of description and do not limit the positional relationship.

As used herein, “substantially” or “approximately” or “about” means not only the stated value, but also the mean within an acceptable range of deviations, considering the errors associated with the corresponding measurement and the measurement of the measured value. For example, “substantially” or “approximately” can mean within ±10%, 5%, 3%, or 1% or within standard deviation of the stated value.

As used herein, when a definition is not otherwise provided, “combination” refers to a mixture of two or more, substitution in which one substituent is substituted with another substituent, fusion with each other, or a linkage to each other by a single bond or a C1 to C1 0 alkylene group.

As used herein, when a definition is not otherwise provided, “combination” may mean a mixture of two or more, an alloy of two or more, and a stacked structure of two or more.

As used herein, when a definition is not otherwise provided, “substituted” refers to replacement of a hydrogen atom of a compound by at least one substituent of halogen, a hydroxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a C1 to C30 alkyl group, a C2 to C30 alkenyl group, a C2 to C30 alkynyl group, a C6 to C30 aryl group, a C7 to C30 arylalkyl group, a C1 to C30 alkoxy group, a C1 to C20 heteroalkyl group, a C3 to C20 heterocyclic group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, and any combination thereof.

As used herein, when a definition is not otherwise provided, “hetero” refers to one including 1 to 4 heteroatoms of N, O, S, Se, Te, Si, or P.

As used herein, when a definition is not otherwise provided, “alkyl group” may be a C1 to C30 (e.g., C1 to C20) linear or branched, saturated, monovalent hydrocarbon group (e.g., a methyl group, an ethyl group, a propyl group, an isobutyl group, a sec-butyl group, a tert-butyl group, a pentyl group, an iso-amyl group, a hexyl group, and the like).

As used herein, when a definition is not otherwise provided, “alkoxy group” may refer to a C1 to C30 (e.g., C1 to C20) alkyl group that is linked via an oxygen, e.g., a methoxy group, an ethoxy group, and a sec-butoxy group.

As used herein, when a definition is not otherwise provided, “aryl group” refers to a monovalent functional group formed by the removal of one hydrogen atom from one or more rings of a C6 to C30 (e.g., C6 to C20) arene, e.g., a phenyl group or a naphthyl group. The arene refers to a hydrocarbon having an aromatic ring, and includes monocyclic and polycyclic hydrocarbons wherein the additional ring(s) of the polycyclic hydrocarbon may be aromatic or nonaromatic.

As used herein, when a definition is not otherwise provided, “heteroaryl group” includes at least one heteroatom such as N, 0, S, Se, Te, Si, or P in the aforementioned aryl group includes at least one heteroatom such as N, O, S, Se, Te, Si, and P and the remaining carbon.

As used herein, when a definition is not otherwise provided, “aralkyl group” may refer to a group represented by —(CR^{2a}R^{2b})ₘAr, wherein R^{2a} and R^{2b} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group, and Ar is the aforementioned aryl group or heteroaryl group.

As used herein, when a definition is not otherwise provided, “heterocyclic group” includes at least one heteroatom such as N, O, S, Se, Te, Si, or P in a ring such as a C6 to C30 (e.g., C6 to C20) aryl group, a C3 to C30 (e.g., C3 to C20) cycloalkyl group, a fused ring thereof, or any combination thereof, and the remaining carbon. When the heterocyclic group is a fused ring, a heteroatom may be included in the entire heterocyclic group or at least one of the rings.

As used herein, when a definition is not otherwise provided, “aromatic ring” refers to a functional group in which all atoms in the cyclic functional group have a pi-orbital, and wherein these pi-orbitals are conjugated. For example, the aromatic ring may be a (e.g., C6 to C20) C6 to C30 aryl group or C3 to C30 (e.g., C3 to C20) heteroaryl group.

Hereinafter, a device, layer, polymer semiconductor, element, region, or the like that is described as being “stretchable” will be understood to be elastic and/or configured to be elastic, such that the device, layer, polymer semiconductor, element, region, or the like is configured to be elastically deformed (e.g., stretched, compressed, subjected to strain, etc.) such that the device, layer, polymer semiconductor, element, region, or the like is configured to resume its same original shape after being deformed. For example, a stretchable device, layer, polymer semiconductor, element, region, or the like as described herein may be capable of being elastically deformed such that the stretchable device, layer, polymer semiconductor, element, region, or the like can resume, and does resume, an original shape after being stretched or compressed. Hereinafter, a device, layer, polymer semiconductor, element, region, or the like that is described as being “non-stretchable” or “rigid” will be understood to be non-elastic and/or not configured to be elastic, such that the device, layer, element, region, or the like is configured to not be elastically deformed (e.g., stretched, compressed, subjected to strain, etc.) such that the device, layer, polymer semiconductor, element, region, or the like is configured to not resume its same original shape after being deformed. For example, a non-stretchable device, layer, polymer semiconductor, element, region, or the like as described herein may not be able to be elastically deformed due to applied strain such that the non-stretchable device, layer, polymer semiconductor, element, region, or the like cannot, and does not, resume an original shape after being stretched or compressed.

Hereinafter, a polymer according to some example embodiments is described.

The polymer according to some example embodiments may be a stretchable polymer, or may be a copolymer including a first structural unit and a second structural unit. The first structural unit may be a semiconducting structural unit including an electron accepting moiety (—L¹—A¹—L² —, wherein A¹ is a diketopyrrolopyrrole structural unit) and an electron donating moiety (—D¹—) and the second structural unit may be a structural unit including an electron accepting moiety (—L¹—A¹—L² —, wherein A¹ is a diketopyrrolopyrrole structural unit) and an electron donating moiety (—D²—). An electron accepting moiety may be optionally included in the second structural unit. The substituents (R¹¹ and R¹²) of the diketopyrrolopyrrole and L¹ and L² in the first structural unit and the second structural unit may be the same or different from each other. The polymer may exhibit semiconductor characteristics through an interaction between an electron donating moiety and an electron accepting moiety.

The first structural unit includes an electron accepting moiety —L¹—A¹—L²—) and an electron donating moiety (—D¹—) may provide improved electrical properties to the polymer, and high charge mobility due to relatively high crystallinity, and the second structural unit includes an electron donating moiety (—D²—) having an asymmetric side chain (—OR²¹ and —R²²) to reduce the crystallinity of the polymer and to improve stretchability. In the second structural unit, —OR²¹, one of the asymmetric side chains, improves the solubility of the polymer, and —R²², the other one of the asymmetric side chains, is a bulky side chain that induces the formation of an amorphous domain to improve stretchability. In addition, since the second structural unit has a fused ring of a conjugated structure, electrical properties of a polymer including the second structural unit are improved, and thus may be usefully applied to an active layer of a transistor.

In some example embodiments, a polymer including a first structural unit represented by Chemical Formula 1 and a second structural unit represented by Chemical Formula 2 is provided.

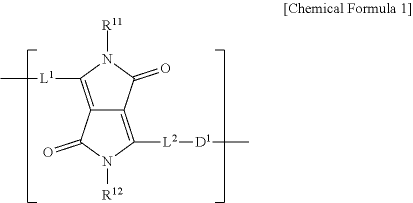

In Chemical Formula 1,
R¹¹ and R¹² may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, —COR^{a}, —OC(═O)R^{b}, —C(═O)OR^{c}, —OC(═O)OR^{d}, a halogen, a cyano group, or any combination thereof, wherein R a to R d may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, or a cyano group,
L¹ and L² may each independently be a single bond, a substituted or unsubstituted C6 to C30 arylene group, a substituted or unsubstituted divalent C3 to C30 heterocyclic group, a fused ring thereof, or any combination thereof, and
D¹ may be a substituted or unsubstituted C6 to C30 arylene group; a substituted or unsubstituted divalent C3 to C30 heterocyclic group including at least one of N, O, S, Se, Te. and Si; a fused ring thereof; or any combination thereof.

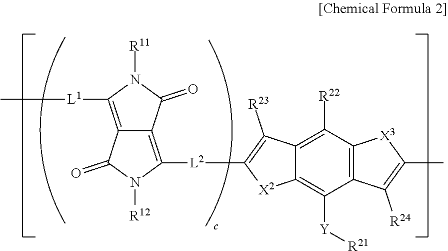

wherein, in Chemical Formula 2,
R¹¹ and R¹² may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, —COR^{a}, —OC(═O)R^{b}, —C(═O)OR^{b}, —OC(═O)OR^{d}, a halogen, a cyano group, or any combination thereof, wherein R^{a} to R^{d} may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, a cyano group, or any combination thereof,

L¹ and L² may each independently be a single bond, a substituted or unsubstituted C6 to C30 arylene group, a substituted or unsubstituted divalent C3 to C30 heterocyclic group, a fused ring thereof, or any combination thereof,
c is 0 or 1,
Y is O, C(═O), —OC(═O), —C(═O)O, or —OC(═O)O,
R²¹ is a substituted or unsubstituted C1 to C30 linear or branched alkyl group, a C3 to C30 linear or branched alkyl group in which at least one of non-adjacent methylene groups is replaced by oxygen (O), a substituted or unsubstituted C3 to C30 cycloalkyl group, or a substituted or unsubstituted C3 to C30 heterocycloalkyl group,
R²² is a substituted or unsubstituted C6 to C30 aryl group, a substituted or unsubstituted C7 to C30 aralkyl group, a substituted or unsubstituted C2 to C30 heteroaryl group, a substituted or unsubstituted C3 to C30 cycloalkyl group, or a substituted or unsubstituted C3 to C30 heterocycloalkyl group,
R²³ and R²⁴ are each independently hydrogen, a substituted or unsubstituted C1 to C30 linear or branched alkyl group, a substituted or unsubstituted C1 to C30 linear or branched alkoxy group, —COR^{a}, —OC(═O)R^{b}, —C(═O)OR^{b}, —OC(═O)OR^{d}, a halogen, or a cyano group, wherein R^{a} to R^{d} are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, or a cyano group, and
X² and X³ are each independently O, S, Se, or Te.

For example, in Chemical Formula 1, R¹¹ and R¹² may each be a relatively long linear alkyl group or a bulky branched alkyl group, and for example, may each independently be a substituted or unsubstituted C6 to C30 linear alkyl group or a substituted or unsubstituted C6 to C30 branched alkyl group, and for example, may each independently be a substituted or unsubstituted C8 to C30 linear alkyl group or a substituted or unsubstituted C8 to C30 branched alkyl group, or a substituted or unsubstituted C10 to C30 linear alkyl group or a substituted or unsubstituted C10 to C30 branched alkyl group. Accordingly, the polymer may have high solubility in an organic solvent.

In Chemical Formula 1, D 1 may be an electron donating moiety, for example, a substituted or unsubstituted C6 to C30 arylene group; a substituted or unsubstituted divalent C3 to C30 heterocyclic group including at least one selected from N, O, S, Se, Te, and Si; a fused ring thereof; or any combination thereof. For example, —D¹— may be at least one substituted or unsubstituted phenylene group; at least one substituted or unsubstituted naphthylene group; at least one substituted or unsubstituted anthracenylene group; at least one substituted or unsubstituted phenanthrenylene group; at least one substituted or unsubstituted pentagonal ring group including at least one selected from N, O, S, Se, Te, and Si; a fused ring of two or more of the above substituted or unsubstituted pentagonal ring groups; a fused ring of at least one substituted or unsubstituted pentagonal ring group and at least one substituted or unsubstituted phenylene group; a fused ring of at least one the substituted or unsubstituted pentagonal ring group and at least one substituted or unsubstituted naphthylene group; a fused ring of at least one the substituted or unsubstituted pentagonal ring group and at least one substituted or unsubstituted anthracenylene group; a fused ring of at least one the substituted or unsubstituted pentagonal ring group and at least one substituted or unsubstituted phenanthrenylene group; or any combination thereof.

In Chemical Formula 1, -D 1 - may be, for example, one of the electron donating moieties listed in Group 1, but is not limited thereto.

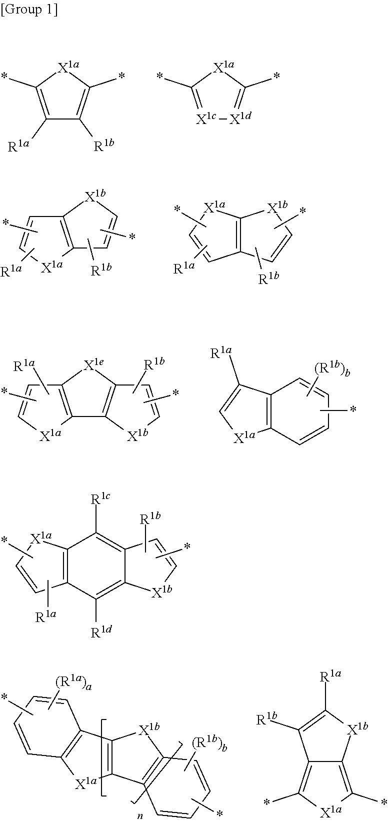

In Group 1,
X^{1a} and X^{1b} are each independently O, S, Se, or Te,
X^{1c} and X^{1d} are each independently N, CR^{x}, or SiR^{y},
X^{1e} is O, S, Se, Te, NR^{v}, CR^{w}R^{x}, or SiR^{y}R^{z},
R^{1a}, R^{1b}, R^{1c}, R^{v}, R^{w}, R^{x}, R^{y}, and R^{z} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
a and b are each independently an integer ranging from 1 to 4,
n is 0, 1, or 2, and
* is a linking point.

In Chemical Formula 1, L¹ and L² may each independently be a single bond; one of electron donating moieties listed in Group 1; a substituted or unsubstituted pyridine; a substituted or unsubstituted pyrimidine; a fused ring thereof; or any combination thereof.

For example, L¹ and L² may each independently be a divalent linking group including a single bond; at least one substituted or unsubstituted furan; at least one substituted or unsubstituted thiophene; at least one substituted or unsubstituted selenophene; at least one substituted or unsubstituted tellurophene; at least one substituted or unsubstituted pyrrole; at least one substituted or unsubstituted benzene; at least one substituted or unsubstituted pyridine; at least one substituted or unsubstituted pyrimidine; or a fused ring of two or more selected from the foregoing groups; or any combination thereof. For example, L¹ and L² may each be different from D¹.

For example, L¹ and L² may be the same as or different from each other.

In Chemical Formula 2, R²² may be one of moieties represented by Group 2.

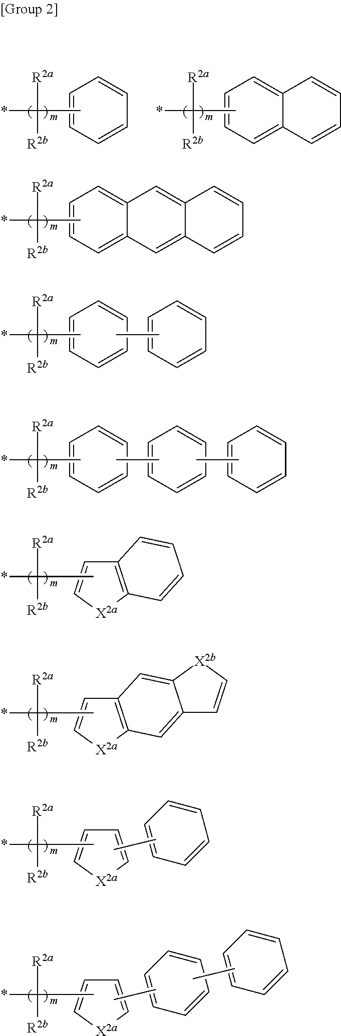

In Group 2,
X² a and X² b are each independently O, S, Se, or Te,
R^{2a} and R^{2b} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
m is an integer ranging from 0 to 10, and
* is a linking point.

In Group 2, a hydrogen atom of the benzene ring may be substituted or unsubstituted with a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group.

In Group 2, at least one (CH₂) in the benzene ring may be replaced by nitrogen (N). That is, in Group 2, the benzene ring may be replaced by a pyridine ring, a pyrimidine ring, a pyrazine ring, or a triazine ring, or two or more benzene rings may each independently be replaced by a pyridine ring, a pyrimidine ring, a pyrazine ring, or a triazine ring.

In —(CR^{2a}R^{2b})ₘ— of Group 2, when m is 2 or more and less than or equal to 10, —CR^{2a}R^{2b}— that are not adjacent to each other may be replaced by —O—, —S—, —C(═O)—, —OC(═O)—, —C(═O)O—, —OC(═O)O—, —S(═O)—, —S(═O)₂—, or any combination thereof.

In some example embodiments, X² and X³ in Chemical Formula 2 may be the same as or different from each other. In some example embodiments, X² and X³ may be S, Se, or Te.

In Chemical Formula 2, R²¹, R²³, and R²⁴ independently may be a substituted or unsubstituted C6 to C30 linear alkyl group or a substituted or unsubstituted C6 to C30 branched alkyl group. Accordingly, the polymer may have high solubility in organic solvents.

The polymer may be, for example, a random copolymer, a block copolymer or an alternating copolymer.

In the polymer including the first structural unit represented by Chemical Formula 1 and the second structural unit represented by Chemical Formula 2, an aspect ratio (Z/X) obtained by dividing the shortest axis length (Z) by the longest axis (X) length of a compound including (e.g., composed of) one first structural unit and one second structural unit may be less than or equal to about 1.4, less than or equal to about 1.3, or less than or equal to about 1.2. The polymer including structural units providing an aspect ratio within the above range may maintain planarity excellently and improve charge mobility.

Each number of the first structural unit and the second structural unit in the polymer may be 1 to 1000, 1 to 800, 2 to 1000, 2 to 800, 5 to 800, 5 to 700, 5 to 500, or 5 to 300, but is not limited thereto. For example, a sum of the number of the first structural unit and the second structural unit in the polymer may not exceed 2000.

The first structural unit and the second structural unit may be included in a molar ratio of about 1:9 to about 9:1, within the above range, for example, about 2:8 to about 8:2, about 3:7 to about 7:3, about 4:6 to about 6:4, or about 5:5. Within the above range, electrical properties such as charge mobility and stretchability may be implemented at the same time.

For example, the first structural unit may be included in a molar ratio of greater than or equal to that of the second structural unit, and for example the first structural unit and the second structural unit may be included in a molar ratio of about 5:5 to about 9:1, about 6:4 to about 9:1, and about 7:3 to about 9:1 or about 8:2 to about 9:1.

For example, the second structural unit may be included in a molar ratio greater than or equal to that of the first structural unit, and for example the first structural unit and the second structural unit may be included in a molar ratio of about 1:9 to about 5:5, about 2:8 to about 5:5, or about 3:7 to about 5:5.

In some example embodiments, the first structural unit may be included in an amount of greater than about 50 mol %, greater than or equal to about 55 mol %, greater than or equal to about 60 mol %, greater than or equal to about 65 mol %, greater than or equal to about 70 mol %, greater than or equal to about 75 mol %, or greater than or equal to about 80 mol % and less than or equal to about 90 mol %, less than or equal to about 89 mol %, less than or equal to about 88 mol %, less than or equal to about 87 mol %, less than or equal to about 86 mol %, or less than or equal to about 85 mol % based on the total amount of the first structural unit and the second structural unit.

In some example embodiments, a molar ratio of D 1 of the first structural unit and D² of the second structural unit may be in the range of about 7:3 to about 9:1 or about 8:2 to about 9:1. Within the above range, it is possible to simultaneously secure high planarity and stretchability of the polymer.

The polymer may further include additional structural units different from the first structural unit and the second structural unit in addition to the aforementioned first structural unit and second structural unit. In some example embodiments, the additional structural unit may be a structural unit composed of —D¹— included in the first structural unit and/or a structural unit composed of —D²— included in the second structural unit.

The polymer may include, for example, structural units represented by Chemical Formulas 3A to 3D, but is not limited thereto.

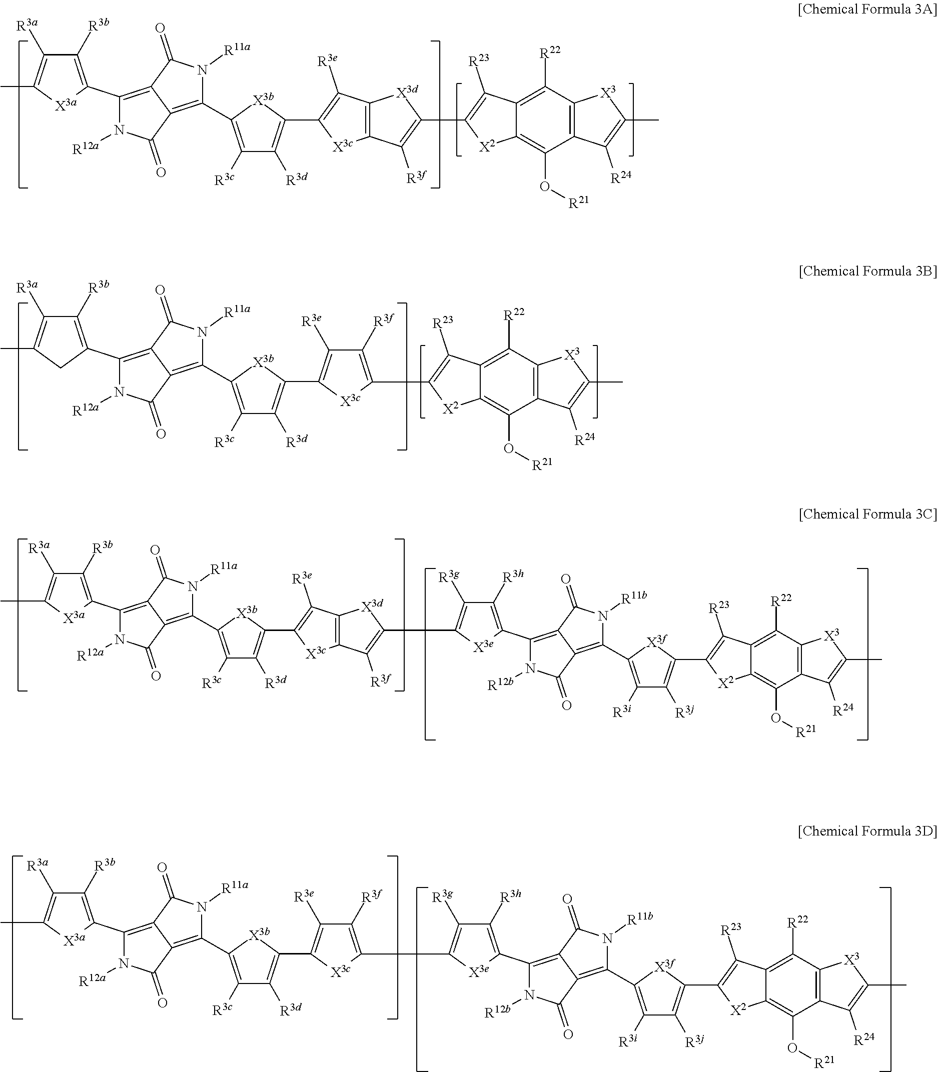

In Chemical Formulas 3A to 3D,
X^{3a}, X^{3b}, X^{3c}, X^{3d}, X^{3e}, X^{3f}, X², and X³ are each independently O, S, Se, or Te,
R^{3a}, R^{3b}, R^{3c}, R^{3d}, R^{3e}, R^{3f}, R^{3g}, and R^{3h} may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
R^{11a}, R^{12a}, R^{11b}, R^{12b}, R²³, and R²⁴ may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, —COR^{a}, —OC(═O)R^{b}, —C(═O)OR^{c}, —OC(═O)OR^{d}, a halogen, a cyano group, or any combination thereof, wherein R^{a} to R^{d} may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, or a cyano group,
R²¹ may be a substituted or unsubstituted C1 to C30 linear or branched alkyl group, a C3 to C30 linear or branched alkyl group in which at least one of non-adjacent methylene groups is replaced by oxygen (O), a substituted or unsubstituted C3 to C30 cycloalkyl group, or a substituted or unsubstituted C3 to C30 heterocycloalkyl group, and
R²² may be a substituted or unsubstituted C6 to C30 aryl group, a substituted or unsubstituted C7 to C30 aralkyl group, a substituted or unsubstituted C2 to C30 heteroaryl group, a substituted or unsubstituted C3 to C30 cycloalkyl group, or a substituted or unsubstituted C3 to C30 heterocycloalkyl group.

Other structures (moieties) of Group 1 examples as electron donating moieties in the structural units may be included in the structures of Chemical Formulas 3A to 3D.

The polymer may have a structure of —L¹-A¹—L²—D¹—D²— as in Chemical Formulas 3A and 3B, and —L¹—A¹—L²—D¹—L¹—A¹—L²—D²— as in Chemical Formulas 3C and 3D. As in the above structure, charge mobility and stretchability may be simultaneously secured by including the different electron donating structures (D¹ and D²).

A terminal end of the polymer may be capped with an aryl group such as a phenyl group.

A weight average molecular weight of the polymer may be greater than or equal to about 5,000 Da, greater than or equal to about 10,000 Da, greater than or equal to about 15,000 Da, greater than or equal to about 20,000 Da, and greater than or equal to about 500,000 Da, and less than or equal to about 450,000 Da, less than or equal to about 400,000 Da, less than or equal to about 350,000 Da, less than or equal to about 300,000 Da, or less than or equal to about 200,000 Da.

The polymer has excellent electrical properties and flexibility and thus may be used as an organic semiconductor material.

The organic semiconductor material may further include the polymer according to some example embodiments and any additional components required for preparing the organic semiconductor from the polymer. For example, such additional components may be a solvent, a binder, an elastomer, etc. for dissolving the polymer, and may further include additional components for imparting additional characteristics to the organic semiconductor produced.

The solvent may include, for example, any solvent capable of dissolving and/or dispersing the polymer according to some example embodiments and not reacting with the polymer. Such a solvent may be any solvent generally used for dissolving a polymer in the technical field to which example embodiments pertain, and may be for example, any organic solvent having a relatively low boiling point which is heated and removed at a low temperature and leaves little solvent residue. Examples of such solvents may be, for example, an aromatic or aliphatic hydrocarbon, a halogenated hydrocarbon, an ester, or an ether amide solvent, and specific examples thereof may include, for example, chloroform, tetrachloroethane, tetrahydrofuran, toluene, tetralin, decalin, anisole, xylene, ethyl acetate, methyl ethyl ketone, dimethyl formamide, chlorobenzene, dichlorobenzene, trichlorobenzene, propylene glycol monomethyl ether acetate (PGMEA), and any combination thereof, but is not limited thereto. In some example embodiments, the solvent may include xylene, toluene, tetralin, decalin, chloroform, chlorobenzene, ortho-dichlorobenzene, trichlorobenzene, or any combination thereof, but is not limited thereto.

The binder may improve dispersibility of the polymer according to some example embodiments, and may include, for example, polystyrene, etc., but is not limited thereto.

The elastomer may be mixed with a polymer according to some example embodiments to improve stretchability of an organic semiconductor prepared therefrom, and may include, for example, an organic elastomer, an organic-inorganic elastomer, an inorganic elastomer-like material, or any combination thereof. Examples of the organic elastomer or organic/inorganic elastomer may include substituted or unsubstituted polyorganosiloxane such as polydimethylsiloxane; an elastomer including a substituted or unsubstituted butadiene moiety such as styrene-ethylene-butylene-styrene (SEBS), styrene-ethylene-propylene-styrene (SEPS), styrene-butadiene-styrene (SBS), styrene-isoprene-styrene (SIS), and styrene-isobutylene-styrene (SIBS); an elastomer containing a urethane moiety; an elastomer including an acrylic moiety; an elastomer including an olefin moiety, or any combination thereof, but are not limited thereto. The inorganic elastomer-like material may include, but is not limited to, ceramic, solid metal, liquid metal, or any combination thereof having elasticity.

The organic semiconductor material may be coated on a substrate or the like in a solution and/or suspension state to be manufactured into a thin film. The method of coating the solution and/or suspension on the substrate may include any method known in the art, for example spin-coating, dip-coating, screen printing, microcontact printing, doctor blading, and the like, but is not limited thereto.

As described above, the organic semiconductor material according to some example embodiments may be coated on a substrate and then dried and/or cured to form a thin film, and the thin film thus produced may be used as, for example, an organic semiconductor.

The aforementioned polymer or organic semiconductor material may be implemented as a thin film. The thin film may be a stretchable polymer thin film. The stretchable polymer thin film can flexibly respond to external forces or external movements such as twisting, pressing and pulling due to the stretching characteristics of the aforementioned polymer and can be easily restored to its original state.

An elastic modulus of the stretchable polymer thin film may be, for example, less than about 10⁷ Pa, and within the above range, for example, greater than or equal to about 10 Pa and less than 10⁷ Pa. For example, an elongation rate of the stretchable polymer thin film may be greater than or equal to about 10%, and within the above range, about 10% to about 1000%, about 10% to about 800%, about 10% to 500%, about 10% to about 300%, about 10% to about 200%, about 10% to about 100%, about 10% to about 90%, about 10% to about 80%, about 10% to about 70%, about 10% to about 60%, about 10% to about 50%, about 10% to about 40%, about 20% to about 70%, about 20% to about 60%, about 20% to about 50%, or about 20% to about 40%.

Herein, the elongation rate may be a percentage of a length change that is increased to a breaking point with respect to the initial length. For example, when the stretchable polymer thin film is stretched, a change in electrical properties of the stretchable polymer thin film may be relatively small. For example, when the stretchable polymer thin film is stretched by about 30%, the charge mobility change of the stretchable polymer thin film may be less than or equal to about 10%, less than or equal to about 8%, less than or equal to about 7%, less than or equal to about 5%, less than or equal to about 3%, less than or equal to about 2%, or less than or equal to about 1%.

The stretchable polymer thin film may be a deposited thin film formed by vapor deposition or a coated thin film formed by a solution process. As described above, since polymers and organic semiconductor materials have good solubility in organic solvents, the stretchable polymer thin film may be a coated thin film formed by a solution process.

The stretchable polymer thin film may further include a binder and/or an elastomer in addition to the aforementioned polymer.

The binder may improve dispersibility of the aforementioned polymer, and may be, for example, polystyrene, but is not limited thereto.

The elastomer may be mixed with the aforementioned polymer to provide stretchability, and may include an organic elastomer, an inorganic elastomer, an inorganic elastomer-like material, or any combination thereof. Examples of the organic elastomer or organic/inorganic elastomer may include substituted or unsubstituted polyorganosiloxane such as polydimethylsiloxane; an elastomer including a substituted or unsubstituted butadiene moiety such as styrene-ethylene-butylene-styrene (SEBS), styrene-ethylene-propylene-styrene (SEPS), styrene-butadiene-styrene (SBS), styrene-isoprene-styrene (SIS), and styrene-isobutylene-styrene (SIBS); an elastomer containing a urethane moiety; an elastomer including an acrylic moiety; an elastomer including an olefin moiety, or any combination thereof, but are not limited thereto. The inorganic elastomer-like material may include, but is not limited to, ceramic, solid metal, liquid metal, or any combination thereof having elasticity.

As described above, the stretchable polymer thin film includes the first structural unit providing stretchability and the second structural unit providing good electrical characteristics, so that it can be stretched and have high charge mobility. Therefore, it can be applied to various electronic devices requiring stretchability and high charge mobility.

The electronic device may include, for example, an organic photoelectric device, an organic light emitting device, or an organic diode including an organic sensor; an organic thin film transistor; an attachable device such as a biometric sensor; or a device including them.

The electronic device may be applied to a bendable display panel, a foldable display panel, a rollable display panel, a wearable device, a skin-like display panel, a skin-like sensor, a large-area conformable display, smart clothing, and the like, but is not limited thereto.

Hereinafter, an example of a thin film transistor including the aforementioned polymer, organic semiconductor material, or stretchable polymer thin film will be described with reference to the drawings.

In the drawings, the thickness of layers, films, panels, regions, etc., are exaggerated for clarity. Like reference numerals designate like elements throughout the specification. It will be understood that when an element such as a layer, film, region, or substrate is referred to as being “on” another element, it may be directly on the other element or intervening elements may also be present. In contrast, when an element is referred to as being “directly on” another element, there are no intervening elements present.

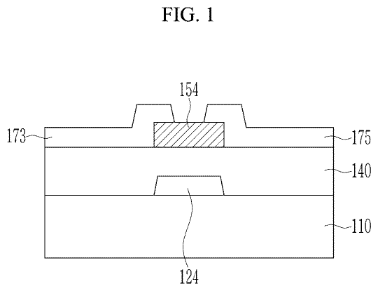

FIGS. 1 to 3 are cross-sectional views each showing a thin film transistor according to some example embodiments.

Referring to FIGS. 1 to 3, a thin film transistor according to an embodiment includes a gate electrode 124, an organic semiconductor 154 overlapped with the gate electrode 124, a gate insulating layer 140 between the gate electrode 124 and the organic semiconductor 154, and a source electrode 173 and a drain electrode 175 electrically connected to the organic semiconductor 154.

First, referring to FIG. 1, a thin film transistor according to some example embodiments may be a thin film transistor having a bottom gate and top contact structure. Specifically, a thin film transistor according to an embodiment includes a gate electrode 124 on the substrate 110; a gate insulating layer 140 on the gate electrode 124; an organic semiconductor 154 on the gate insulating layer 140; and a source electrode 173 and a drain electrode 175 electrically connected to the organic semiconductor 154.

The gate electrode 124 is disposed on a substrate 110 made of transparent glass, silicon, or polymer. The gate electrode 124 is connected to a gate line (not shown) transferring a gate signal. The gate electrode 124 may be made of gold (Au), copper (Cu), nickel (Ni), aluminum (Al), molybdenum (Mo), chromium (Cr), tantalum (Ta), titanium (Ti), an alloy thereof, or a combination of these, but is not limited thereto.

The gate insulating layer 140 is disposed on the gate electrode 124. The gate insulating layer 140 may be made of an organic material or an inorganic material. Examples of the organic material may include a soluble polymer compound such as a polyvinyl alcohol-based compound, a polyimide-based compound, a polyacryl-based compound, a polystyrene-based compound, benzocyclobutane (BCB), styrene-ethylene-butylene-styrene (SEBS), and the like, and examples of the inorganic material may include a silicon nitride (SiNₓ) and silicon oxide (SiO₂).

The organic semiconductor 154 is disposed on the gate insulating layer 140. The organic semiconductor 154 may include the aforementioned polymer or organic semiconductor material, and may be the aforementioned stretchable polymer thin film. The organic semiconductor 154 may be formed by a solution process such as spin coating, slit coating, or inkjet printing by preparing the aforementioned polymer or organic semiconductor material in a solution form. The organic semiconductor 154 may be formed by vacuum evaporation or thermal evaporation of the aforementioned polymer or organic semiconductor material.

The source electrode 173 and drain electrode 175 are disposed on the organic semiconductor 154. The source electrode 173 and the drain electrode 175 face the gate electrode 124 on the organic semiconductor 154 as the center. The source electrode 173 is connected to a data line (not shown) transmitting a data signal. The source electrode 173 and the drain electrode 175 may include at least one metal selected from gold (Au), copper (Cu), nickel (Ni), aluminum (Al), molybdenum (Mo), chromium (Cr), tantalum (Ta), titanium (Ti), an alloy thereof, or any combination thereof, but is not limited thereto.

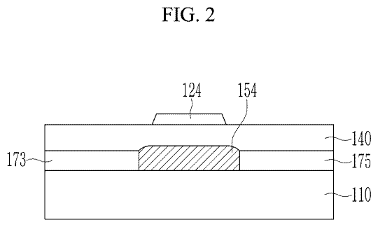

Referring to FIG. 2, the thin film transistor according to an embodiment may be a thin film transistor having a top gate and top contact structure, unlike the aforementioned embodiment. Specifically, a thin film transistor according to an embodiment includes an organic semiconductor 154 on the substrate 110; a source electrode 173 and a drain electrode 175 electrically connected to the organic semiconductor 154; a gate insulating layer 140 on the organic semiconductor 154, the source electrode 173, and the drain electrode 175; and a gate electrode 124 on the gate insulating layer 140.

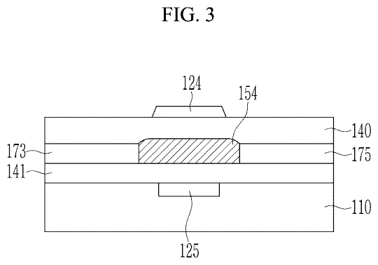

Referring to FIG. 3, the thin film transistor according to an embodiment may be a thin film transistor having a dual gate and bottom/top contact structure, unlike the aforementioned embodiment. Specifically, a thin film transistor according to an embodiment includes an organic semiconductor 154 on the substrate 110; a first gate electrode 125 positioned under the organic semiconductor 154; a second gate electrode 124 positioned on the organic semiconductor 154; a first gate insulating layer 141 between the organic semiconductor 154 and the first gate electrode 125; a second gate insulating layer 140 between the organic semiconductor 154 and the second gate electrode 124; and a source electrode 173 and a drain electrode 175 electrically connected to the organic semiconductor 154. The first gate electrode 125 may be buried in the substrate 110 or may be formed by impurity doping. The first gate electrode 125, the organic semiconductor 154, and the second gate electrode 124 may be overlapped with each other.

Herein, examples of the thin film transistor have been described, but example embodiments are not limited thereto and may be equally applied to thin film transistors having all structures.

The thin film transistor according to example embodiments may be applied to various electronic devices as a switching device and/or a driving device. The electronic devices include, for example, a liquid crystal display device, an organic light emitting display device, a quantum dot display device, an electrophoretic display device, an organic photoelectric device, and an organic sensor, but is not limited thereto. The electronic device including the thin film transistor may be, for example, a flexible and stretchable electronic device, and may be a wearable device and/or a skin type device.

Hereinafter, the embodiments are illustrated in more detail with reference to examples. However, the following examples are for illustrative purposes and do not limit the scope of the rights.

### Synthesis of Polymers

### Synthesis Example 1: Synthesis of Polymer Including Structural Unit Represented by Chemical Formula A-1

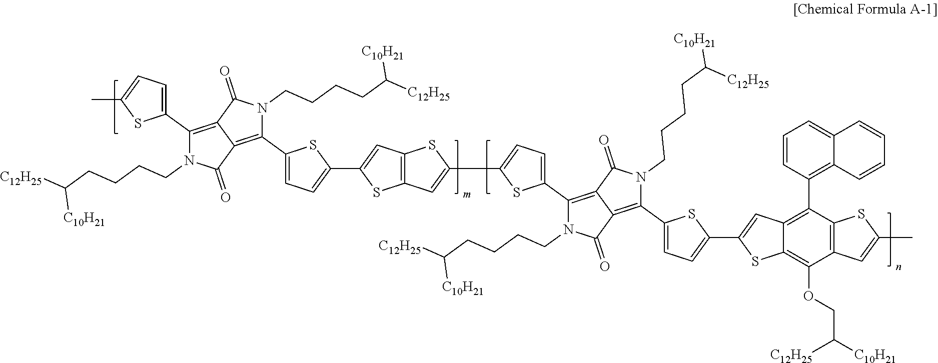

In Chemical Formula A-1, m and n represent a mole ratio of each structural unit, and m:n is 9:1.

Step 1-1: Synthesis of asy-BDT-NaPh

asy-BDT-NaPh is synthesized as follows.

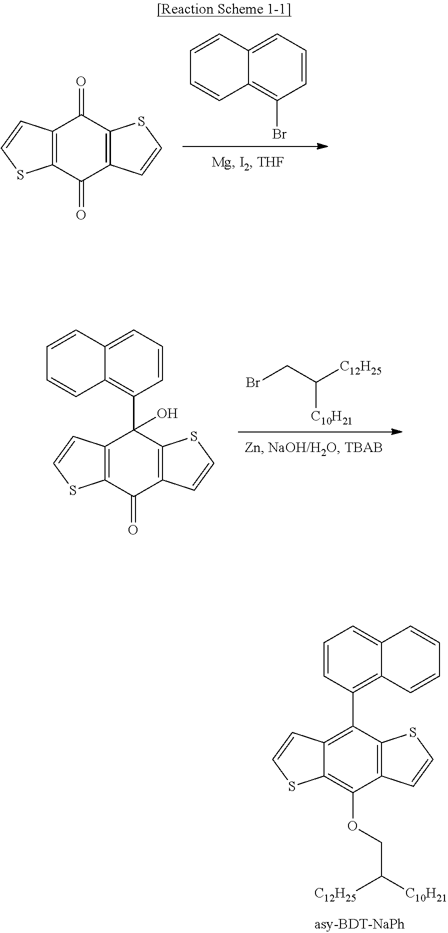

1-bromonaphthalene (1.45 g, 7 mmol) is added to a mixture of magnesium turning (0.22 g, 9 mmol), 12, and dry tetrahydrofuran (dry THF, 10 ml) at a sufficient speed with reflux under nitrogen. After the addition, the obtained mixture is refluxed for 1.5 hours. Subsequently, Grignard reagent is added to a THF solvent (100 ml) in an ice-water bath, and 4,8-dihydrobenzo[1,2-b:4,5-b′]dithiophen-4,8-dione (1.1 g, 5 mmol) is added dropwise thereto. Lastly, the obtained mixture is stirred under a nitrogen atmosphere at room temperature for 6 hours. Subsequently, water is added thereto for hydrolysis, and then, extraction is performed with dichloromethane. Then, drying is performed under Na₂SO₄. After removing the solvents under vacuum, the residue (1.05 g) is used in the next step with further purification.

Compound 1 (0.52 g) and zinc powder (0.26 g, 4 mmol) are put in a 100 ml flask under a nitrogen atmosphere, and NaOH (2 g) (+water (25 ml)) is added thereto. The obtained mixture is well stirred and heated under reflux for 3 hours. Subsequently, 1-bromo-2-decyltetradecane (1.04 g, 2.5 mmol) and tetrabutylammonium bromide (96.7 mg, 0.3 mmol) are added to the flask. After refluxing overnight, the reactant is poured into cold water and extracted with hexane. Then, drying is performed under Na₂SO₄. After removing the solvents under vacuum, the residue is purified through silica column chromatography with hexane/dichloromethane (5/1, v/v), obtaining a compound, asy-BDT-NaPh (light yellow oil, 0.59 g, 35%).

¹H NMR (500 MHz, CDCl₃): δ (ppm) 7.97-7.91 (m, 2H), 7.63-7.56 (m, 3H), 7.48-7.43 (m, 1H), 7.38 (d, J=8.5 Hz, 1 H), 7.30-7.24 (m, 3H), 6.78 (d, J=5.5 Hz, 1 H), 4.35 (d, J=5.5 Hz, 2H), 1.98-1.90 (m, 1 H), 1.73-1.65 (m, 2H), 1.58-1.25 (m, 38H), 0.90-(m, 6H).

¹³C NMR (125 MHz, CDCl₃): δ (ppm) 148.44, 140.78, 139.19, 136.60, 133.94, 131.55, 129.73, 128.61, 128.33, 128.14, 127.82, 126.81, 126.26, 126.13, 126.01, 125.59, 124.54, 123.31, 119.92, 76.35, 39.30, 31.95, 31.38, 30.11, 29.73, 29.71, 29.69, 29.39, 27.04, 22.71, 14.14.

Step 1-2: Synthesis of asy-BDT-NaPh-Sn

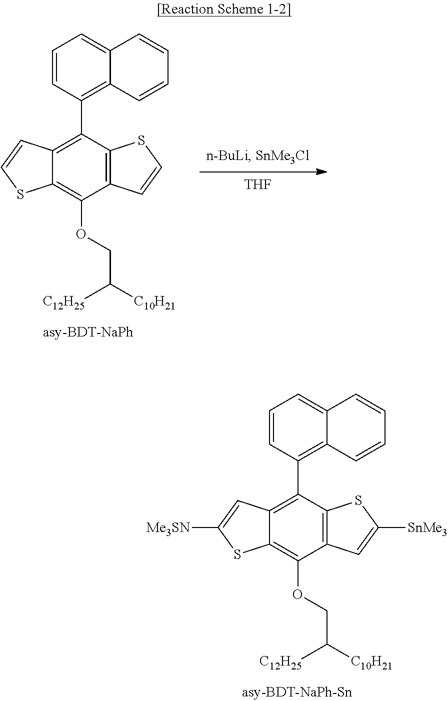

The asy-BDT-NaPh (0.34 g, 0.5 mmol) synthesized in the step 1-1 is added to THF (30 ml) at −78 ° C., and n-BuLi (0.56 mL, 1.4 mmol, 2.5 M in hexane) is added dropwise thereto. After stirring the mixture for 1 hour at −78 ° C., the temperature is increased to room temperature for 0.5 hours. After cooling again to −78 ° C., trimethyltin chloride (1.5 mL, 1.5 mmol, 1 M in hexane) is added thereto. The obtained mixture is heated up to room temperature and stirred overnight. Subsequently, water is added to the reaction mixture, and then, extraction is performed with hexane. Then, after drying with Na₂SO₄ and removing the solvents under a reduced pressure, recrystallization is conducted with ethanol. asy-BDT-NaPh-Sn as an off-white solid is obtained. (0.39 g, 78%).

¹H NMR (500 MHz, CDCl₃): δ (ppm) 8.01-7.95 (m, 2H), 7.66-7.60 (m, 3H), 7.51-7.47 (m, 1H), 7.42 (d, J=8.0 Hz, 1H), 7.31-7.27 (m, 1H), 6.85 (s, 1H), 4.36 (d, J=5.5 Hz, 2H), 1.98-1.91 (m, 1H), 1.76-1.68 (m, 2H), 1.58-1.26 (m, 38H), 0.90-0.86 (m, 6H), 0.41-0.25 (m, 18H).

¹³C NMR (125 MHz, CDCl₃): δ (ppm) 147.03, 145.17, 141.48, 140.92, 140.42, 137.37, 133.90, 132.02, 131.69, 131.02, 130.84, 128.35, 128.23, 127.86, 127.72, 126.36, 126.03,125.97, 125.66, 122.95, 75.98, 39.30, 31.95, 31.94, 31.47, 30.23, 29.81, 29.79, 29.76, 29.74, 29.71, 29.69, 29.40, 29.38, 27.12, 22.70, 14.13, −8.32, −8.41.

MS (MALDI-TOF): cacld for C50H76OS2Sn2 [M]+: 994.338; found: 994.238.

### Step 1-3: PDPP-NaPh Polymerization

DPP-Br (3,6-bis(5-bromothiophen-2-yl)-2,5-bis(5-decylheptadecyl)-2,5-dihydropyrrolo[3,4-c]pyrrole-1,4-dione, 182.3 mg, 0.15 mmol, 1.0 eq.), TT-Sn (2,5-bis(trimethylstannyl)thieno[3,2-b]thiophene, 1.0 eq.), asy-BDT-NaPh-Sn (4-((2-decyltetradecyl)oxy)-8-(naphthalen-1-yl)benzo[1,2-b:4,5-b′]dithiophene-2,6-diyl)bis(trimethylstannane), 0.1 eq.), Pd₂(dba)3 (tris(dibenzylideneacetone)dipalladium (0), 4.1 mg, 0.0045 mmol, 0.03 eq.), and P(o-tol)₃ (tri(o-tolyl)phosphine, 8.6 mg, 0.012 mmol, 0.08 eq.) are put in a reaction vial, and chlorobenzene (4.5 ml) is added thereto in a nitrogen-filled glovebox. After sealing the reaction vial, the mixture is reacted in a microwave reactor with a temperature profile of 100° C. for 1 minute, 120° C. for 2 minutes, 140° C. for 5 minutes, 160° C. for 5 minutes, and 180° C. for 60 minutes. After the reaction, the resultant is cooled, trimethylphenyl tin (24.1 mg, 0.1 mmol) is added thereto and then, heated again at 180° C. for 15 minutes. In order to complete end-capping of a polymer, bromobenzene (23.6 mg, 0.15 mmol) is added thereto, and the reaction vessel is lastly heated at 180° C. for 15 minutes in the microwave reactor.

Subsequently, precipitates are formed in methanol, filtered, loaded in a Soxhlet thimble, and then, washed with methanol, acetone, hexane (for 12 hours, respectively). The polymer is finally obtained in chloroform. Subsequently, the chloroform solution is concentrated and then, precipitated in methanol. The precipitates are filtered and dried under high vacuum for 24 hours, obtaining the polymer represented by Chemical Formula A-1. (Yield: 61%)

molecular weight Mn=55.72 KDa, Mw=182.49 KDa, PDI=3.28. Td=370° C. Element analysis found (%): C 74.27, H 9.79, N 2.24 (Ca. 10.2 mol % asy-BDT-NaPh segment).

### Synthesis Example 2: Synthesis of Polymer Including Structural Unit Represented by Chemical Formula A-2

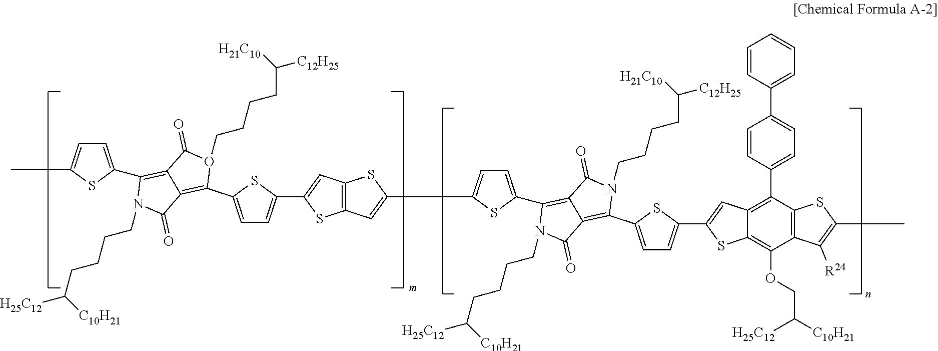

In Chemical Formula A-2, m and n represent a molar ratio of each structural unit, and m:n is 9:1.

Step 2-1: Synthesis of asy-BDT-PhPh

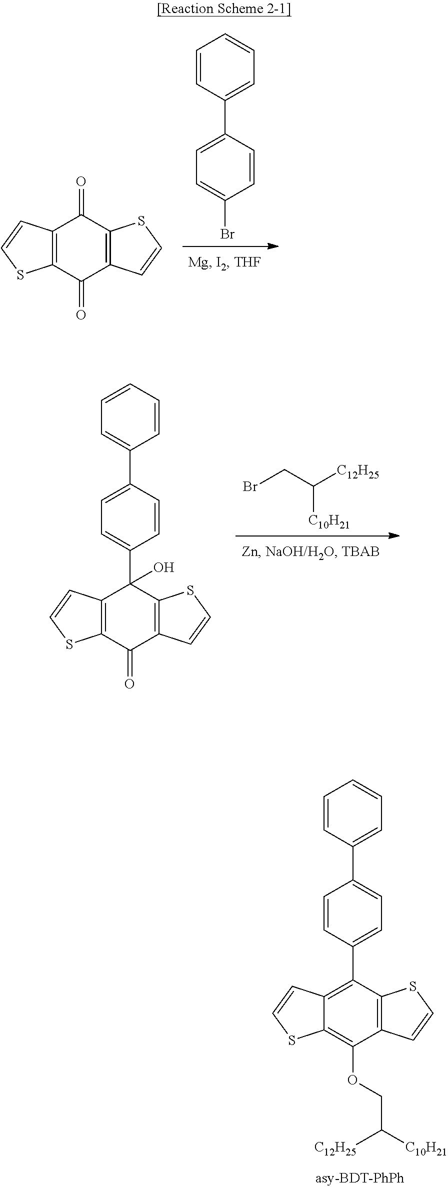

Compound 2 is synthesized by using 4-bromobiphenyl instead of the 1-bromonaphthalene in the step 1-1 of Synthesis Example 1.

Compound 2 (0.96 g) and zinc powder (0.26 g, 4 mmol) are put in a 100 ml flask under a nitrogen atmosphere, and NaOH (2 g) (+water (25 ml)) is added thereto. The obtained mixture is well stirred and then, heated under reflux for 3 hours. Subsequently, 1-bromo-2-decyltetradecane (1.04 g, 2.5 mmol) and tetrabutylammonium bromide (96.7 mg, 0.3 mmol) are added to the flask. After refluxing overnight, the reactant is poured into cold water and then, extracted with hexane. Subsequently, drying is performed under Na₂SO₄. After removing the solvents under vacuum, the residue is purified through silica column chromatography with hexane/dichloromethane (5/1, v/v), obtaining a compound of asy-BDT-PhPh (light yellow oil, 0.49 g, 28%).

¹H NMR (500 MHz, CDCl₃): δ (ppm) 7.78-7.69 (m, 6H), 7.56 (d, J=5.5 Hz, 1 H), 7.51-7.46 (m, 2H), 7.42-7.36 (m, 3H), 7.34 (d, J=5.5 Hz, 1H), 4.28 (d, J=5.5 Hz, 2H), 1.95-1.87 (m, 1H), 1.70-1.63 (m, 2H), 1.55-1.26 (m, 38H), 0.90-0.86 (m, 6H).

¹³C NMR (125 MHz, CDCl₃): δ (ppm) 148.26, 140.72, 140.64, 139.50, 138.25, 138.07, 130.05, 129.88, 128.87, 128.80, 127.47, 127.14, 126.93, 126.21, 126.19, 123.13, 120.14, 76.43, 39.30, 31.97, 31.35, 30.11, 29.74, 29.71, 29.41, 27.03, 22.73, 14.16.

Step 2-2: Synthesis of asy-BDT-PhPh-Sn

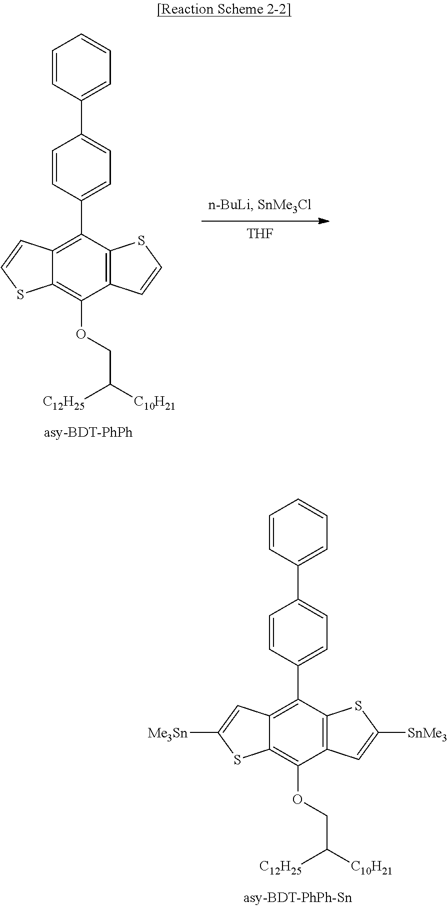

asy-BDT-Ph Ph-Sn (0.28 g, 55%) is synthesized in the same manner as in the step 1-2 of Synthesis Example 1 except that asy-BDT-PhPh is used instead of the asy-BDT-NaPh in the step 1-2 of Synthesis Example 1.

¹H NMR (500 MHz, CDCl₃): δ (ppm) 7.80-7.71 (m, 6H), 7.61 (s, 1H), 7.52-7.46 (m, 2H), 7.41-7.34 (m, 2H), 4.30 (d, J=5.5 Hz, 2H), 1.95-1.88 (m, 1H), 1.74-1.65 (m, 2H), 1.57-1.25 (m, 38H), 0.91-0.86 (m, 6H), 0.55-0.26 (m, 18H).

¹³C NMR (125 MHz, CDCl₃): δ (ppm) 146.85, 143.91, 141.57, 140.80, 140.71, 140.30, 139.24, 138.95, 132.67, 131.12, 130.69, 129.96, 128.81, 127.90, 127.37, 127.12, 124.62, 75.96, 39.29, 31.95, 31.94, 31.44, 30.21, 29.80, 29.78, 29.75, 29.74, 29.71, 29.69, 29.39, 29.38, 27.10, 22.70, 14.13, -8.30, -8.39.

MS (MALDI-TOF): cacld for C52H78OS2Sn2 [M]+: 1020.353; found: 1020.111.

### Step 2-3: PDPP-PhPh Polymerization

A polymer represented by Chemical Formula A-2 in a dark-colored solid state at a yield of 58% is synthesized in the same manner as in the step 1-3 of Synthesis Example 1 except that asy-BDT-PhPh-Sn is used instead of the asy-BDT-NaPh-Sn in the step 1-3 of Synthesis Example 1.

HT-GPC: Mn=55.72 KDa, Mw=182.49 KDa, PDI =3.28. Td=370° C. Element analysis found (%): C 74.27, H 9.79, N 2.24 (Ca. 10.2 mol % asy-BDT-NaPh segment).

### Synthesis Example 3: Synthesis of a Polymer Including Structural Unit Represented by Chemical Formula A-3

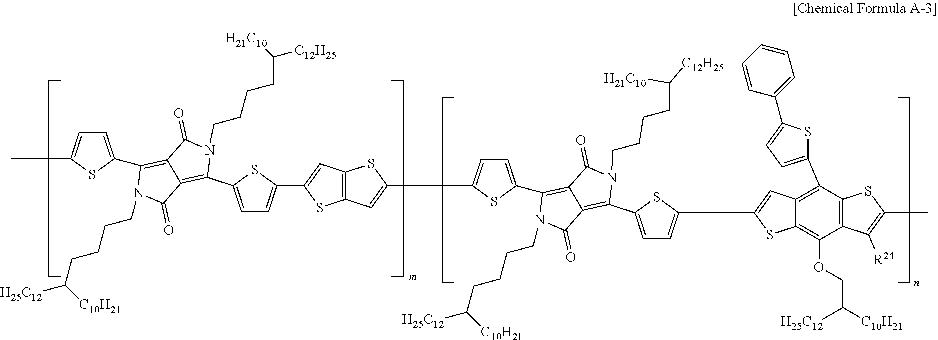

In Chemical Formula A-3, m and n represent a mole ratio of each structural unit, and m:n is 9:1.

Step 3-1: Synthesis of asy-BDT-ThPh

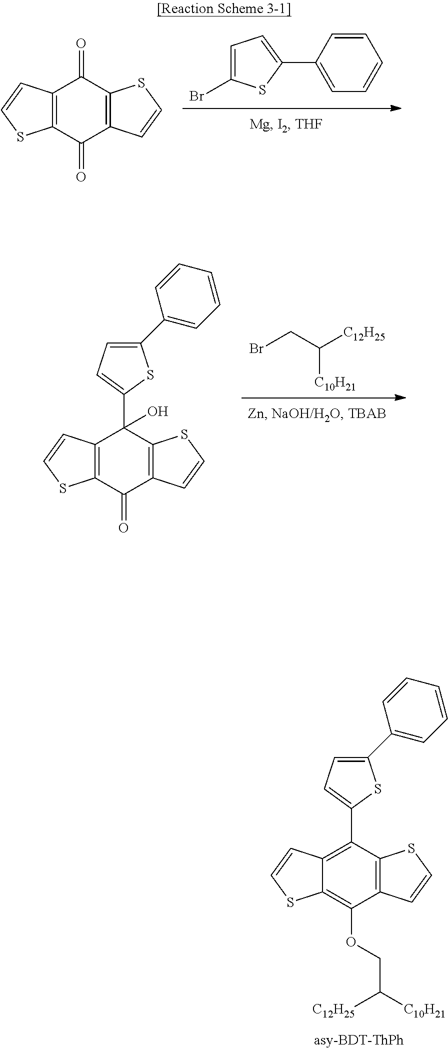

Compound 3 is synthesized in the same manner as in the step 1-1 of Synthesis Example 1 except that 2-bromo-5-phenylthiophene is used instead of the 1-bromonaphthalene.

Compound 3 (1.12 g) and zinc powder (0.26 g, 4 mmol) are put in a 100 ml flask under a nitrogen atmosphere, and NaOH (2 g) (+water (25 ml)) is added thereto. The obtained mixture is well stirred and then, heated under reflux for 3 hours. Subsequently, 1-bromo-2-decyltetradecane (1.04 g, 2.5 mmol) and tetrabutylammonium bromide (96.7 mg, 0.3 mmol) are added to the flask. After refluxing overnight, the reactant is poured into cold water and extracted with hexane. Subsequently, drying is performed under Na₂SO₄. After removing the solvents under vacuum, the residue is purified through silica column chromatography with hexane/dichloromethane (5/1, v/v), obtaining a compound of asy-BDT-ThPh (yellow oil, 0.56 g, 32%).

¹H NMR (500 MHz, CDCl₃): δ (ppm) 7.70-7.65 (m, 2H), 7.63 (d, J=5.5 Hz, 1 H), 7.54 (d, J=5.5 Hz, 1 H), 7.44-7.36 (m, 6H), 7.31-7.27 (m, 1 H), 4.27 (d, J=5.0 Hz, 2H), 1.93-1.85 (m, 1H), 1.69-1.60 (m, 2H), 1.53-1.25 (m, 38H), 0.90-0.85 (m, 6H).

¹³C NMR (125 MHz, CDCl₃): δ (ppm) 148.80, 144.75, 140.54, 139.35, 138.61, 134.24, 130.01, 128.98, 128.83, 128.67, 127.66, 127.33, 126.31, 125.82, 123.42, 123.33, 120.19, 119.15, 76.49, 39.32, 31.99, 31.35, 30.12, 29.78, 29.76, 29.73, 29.44, 27.03, 22.76, 14.19.

Step 3-2: Synthesis of asy-BDT-ThPh-Sn

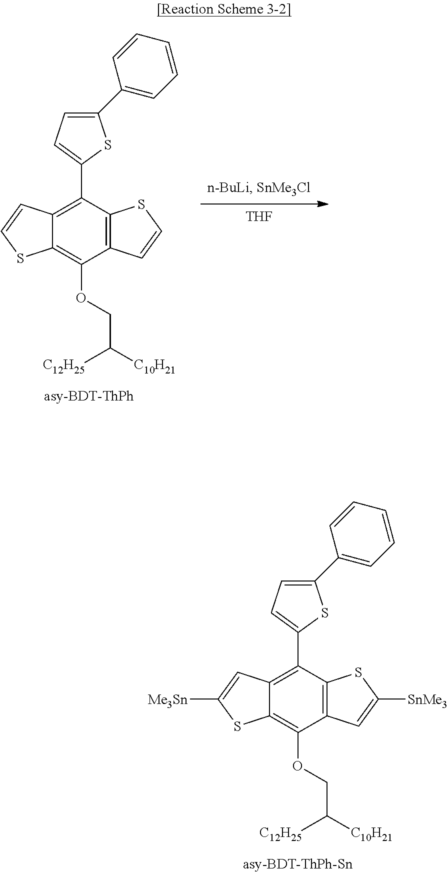

asy-BDT-ThPh-Sn (0.29 g, 57%) of a yellow solid is synthesized in the same manner as in the step 1-2 of Synthesis Example 1 except that asy-BDT-ThPh is used instead of the asy-BDT-NaPh in the step 1-2 of Synthesis Example 1.

1H NMR (500 MHz, CDCl3): δ (ppm) 7.72-7.69 (m, 2H), 7.67 (s, 1H), 7.59 (s, 1 H), 7.45-7.39 (m, 4H), 7.33-7.28 (m, 1H), 4.30 (d, J=5.5 Hz, 2H), 1.94-1.86 (m, 1H), 1.73-1.64 (m, 2H), 1.55-1.25 (m, 38H), 0.90-0.86 (m, 6H), 0.49-0.37 (m, 18H).

13C NMR (125 MHz, CDCl3): δ (ppm) 147.34, 144.92, 144.38, 142.17, 140.91, 140.22, 139.78, 134.38, 132.67, 131.08, 130.93, 128.92, 128.51, 127.91, 127.50, 125.80, 123.29, 117.46, 76.02, 39.28, 31.94, 31.41, 30.19, 29.77, 29.75, 29.74, 29.70, 29.69, 29.39, 29.38, 27.09, 22.70, 14.13, -8.27, -8.36.

MS (MALDI-TOF): cacld for C50H76OS3Sn2 [M]+: 1026.310; found: 1026.200.

### Step 3-3: PDPP-ThPh Polymerization

A polymer represented by Chemical Formula A-3 in a dark-colored solid state is obtained at a yield of 63% in the same manner as in the step 1-3 of Synthesis Example 1 except that asy-BDT-Th Ph-Sn is used instead of the asy-BDT-NaPh-Sn in the step 1-3 of Synthesis Example 1.

### Synthesis Example 4: Synthesis of a Polymer Including Structural Unit Represented by Chemical Formula A-4

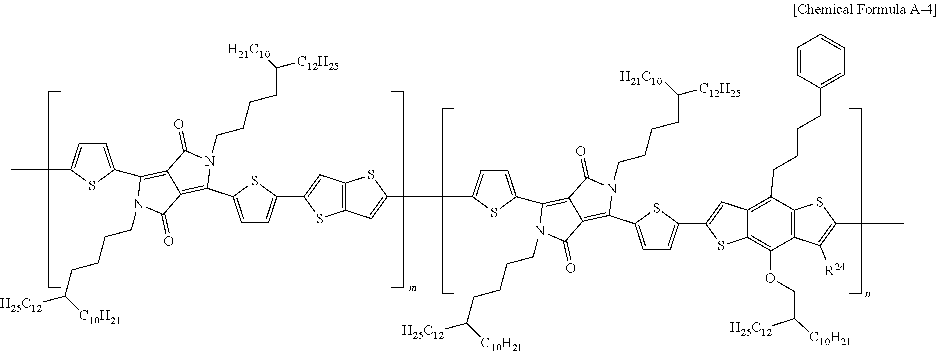

In Chemical Formula A-4, m and n represent a mole ratio of each structural unit, and m:n is 9:1.

Step 4-1: Synthesis of asy-BDT-C4Ph

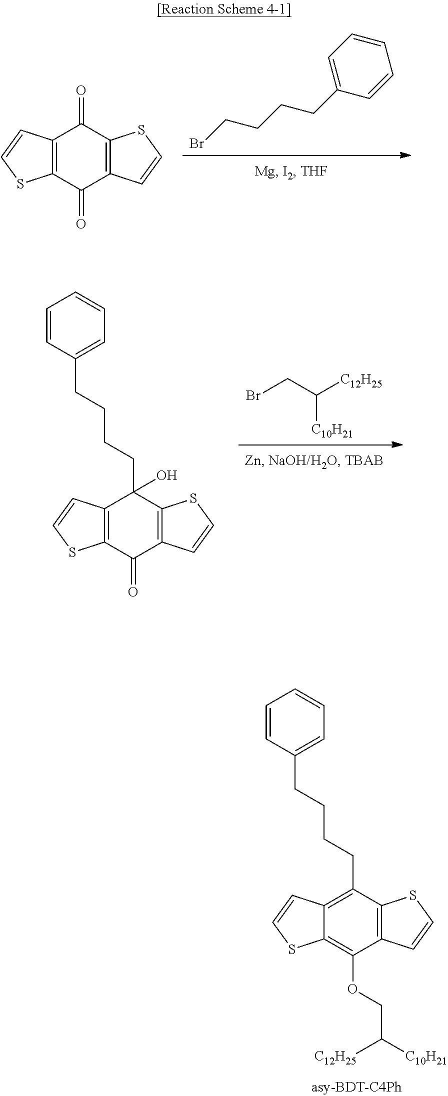

Compound 4 is synthesized in the same manner as in the step 1-1 of Synthesis Example 1 except that 1-bromo-3-phenylbutane is used instead of the 1-bromonaphthalene.

Compound 4 (1.08 g) and zinc powder (0.26 g, 4 mmol) are put in a 100 ml flask under a nitrogen atmosphere, and NaOH (2 g) (+water (25 ml)) is added thereto. The mixture is well stirred and then, heated under reflux for 3 hours. Subsequently, 1-bromo-2-decyltetradecane (1.04 g, 2.5 mmol) and tetraammonium bromide (96.7 mg, 0.3 mmol) are added to the flask. After refluxing overnight, the reactant is poured into cold water and extracted with hexane. Subsequently, drying is performed under Na2SO4. After removing the solvents under vacuum, the residue is purified through silica column chromatography with hexane/dichloromethane (5/1, v/v), obtaining a compound of asy-BDT-C4Ph (colorless oil, 0.64 g, 38%).

¹H NMR (500 MHz, CDCl₃): δ (ppm) 7.45 (d, J=5.5 Hz, 1H), 7.27-7.24 (m, 2H), 7.22 (d, J=5.5 Hz, 1H), 7.19-7.15 (m, 2H), 7.10-7.05 (m, 3H), 4.15 (d, J=5.0 Hz, 2H), 3.09 (t, J=7.5 Hz, 2H), 2.57 (t, J=8.0 Hz, 2H), 1.85-1.75 (m, 3H), 1.73-1.67 (m, 2H), 1.66-1.59 (m, 2H), 1.51-1.23 (m, 38H), 0.89-0.86 (m, 6H).

¹³C NMR (125 MHz, CDCl₃): δ (ppm) 147.19, 142.18, 138.72, 137.79, 129.81, 128.68, 128.30, 128.17, 126.38, 125.80, 125.59, 125.05, 121.52, 120.31, 76.10, 39.26, 35.75, 32.88, 32.00, 31.44, 31.36, 30.14, 29.80, 29.78, 29.75, 29.46, 29.21, 27.04, 22.76, 14.17.

Step 4-2: Synthesis of asy-BDT-C4Ph-Sn

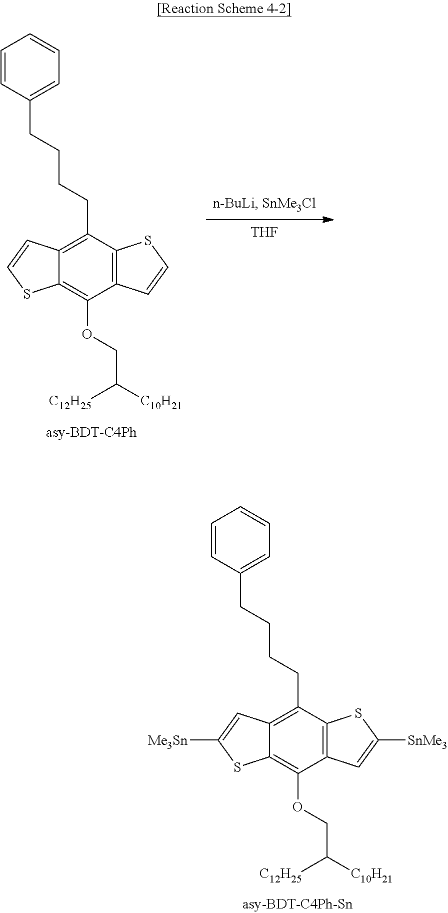

asy-BDT-C4Ph-Sn (0.38 g, 75%) of light-yellow oil is synthesized in the same manner as in the step 1-2 of Synthesis Example 1 except that asy-BDT-C4Ph is used instead of the asy-BDT-NaPh in the step 1-2 of Synthesis Example 1.

¹H NMR (500 MHz, CDCl₃): δ (ppm) 7.56 (s, 1H), 7.43 (s, 1H), 7.27-7.22 (m, 2H), 7.19-7.13 (m, 3H), 4.21 (d, J=5.0 Hz, 2H), 3.22 (t, J=7.5 Hz, 2H), 2.68 (t, J=8.0 Hz, 2H), 1.91-1.84 (m, 3H), 1.82-1.76 (m, 2H), 1.70-1.63 (m, 2H), 1.51-1.25 (m, 38H), 0.90-0.85 (m, 6H), 0.50-0.39 (m, 18H).

¹³C NMR (125 MHz, CDCl₃): δ (ppm) 145.77, 142.47, 140.81, 139.61, 139.05, 132.57, 130.95, 129.39, 128.39, 128.20, 125.59, 124.50, 75.87, 39.23, 35.69, 33.01, 31.93, 31.44, 31.42, 30.19, 29.78, 29.76, 29.74, 29.72, 29.69, 29.68, 29.38, 29.37, 29.22, 27.08, 22.70, 14.13, −8.31, −8.37. MS

(MALDI-TOF): cacld for C50H82OS2Sn2 [M]+: 1000.385; found: 1000.479.

### Step 4-3: PDPP-C4Ph Polymerization

A polymer dark color represented by Chemical Formula A-4 in a solid state at a yield of 57% is synthesized in the same manner as in the step 1-3 of Synthesis Example 1 except that asy-BDT-C4Ph-Sn is used instead of the asy-BDT-NaPh-Sn in the step 1-3 of Synthesis Example 1.

HT-GPC: Mn=52.34 KDa, Mw=167.22 KDa, PDI=3.20. Td=365° C. Element analysis found (%): C 74.57, H 9.88, N 2.24 (Ca. 10.1 mol % asy-BDT-C4Ph segment).

### Comparative Synthesis Example 1: Synthesis of Polymer (PDPPTT Polymer) Including Structural Unit Represented by Chemical Formula B-1

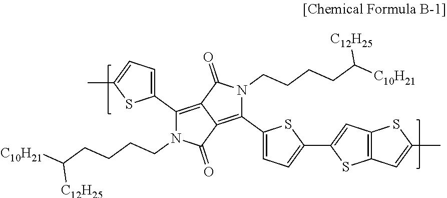

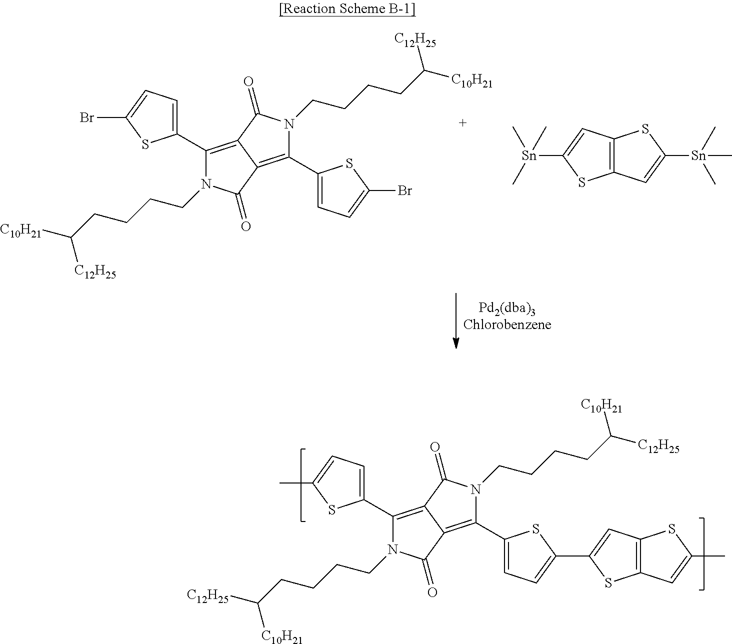

A PDPPTT polymer of a dark-colored solid at a yield of 60% is polymerized in the same manner as in the step 1-3 of Synthesis Example 1 except that the asy-BDT-NaPh-Sn is not used.

HT-GPC: Mn=55.68 KDa, Mw=179.69 KDa, PDI =3.23. Td=391° C. Element analysis found (%): C 74.68, H 9.84, N 2.39.

### Manufacture of Polymer Thin Films

### EXAMPLE 1

The polymer according to Synthesis Example 1 is dissolved in chlorobenzene at a concentration of 5 mg/ml, obtaining a solution. The solution is spin-coated on a SiO₂ substrate surface-treated with octadecyltrimethoxysilane (OTS) and then, heat-treated under a nitrogen atmosphere at 150° C. for 30 minutes, forming a polymer thin film.

### EXAMPLE 2

A polymer thin film is manufactured in the same manner as in Example 1 except that the polymer of Synthesis Example 2 is used instead of the polymer of Synthesis Example 1.

### EXAMPLE 3

A polymer thin film is manufactured in the same manner as in Example 1 except that the polymer of Synthesis Example 3 is used instead of the polymer of Synthesis Example 1.

### EXAMPLE 4

A polymer thin film is manufactured in the same manner as in Example 1 except that the polymer of Synthesis Example 4 is used instead of the polymer of Synthesis Example 1.

### Comparative Example 1

A polymer thin film is manufactured in the same manner as in Example 1 except that the polymer of Comparative Synthesis Example 1 is used instead of the polymer of Synthesis Example 1.

### Manufacture of Thin Film Transistor

### EXAMPLE 5

A substrate doped with Si at high concentration is used as a gate electrode, and a SiO₂ insulating layer (gate insulating layer, 300 nm) is formed on the gate electrode. After a surface treatment with octadecyltrimethoxysilane (OTS), the polymer of Synthesis Example 1 is added to chlorobenzene at a concentration of 0.5 wt % to obtain a polymer solution, and the polymer solution is spin-coated to be 300 Å thick at 1000 rpm and heat-treated at 130° C. for 1 hour under a nitrogen atmosphere to form an active layer (length: 50 μm, width: 1000 μm). On the active layer, Au is thermally deposited to form a source electrode and a drain electrode, manufacturing a thin film transistor.

### EXAMPLE 6

A thin film transistor is manufactured in the same manner as in Example 5 except that the active layer is formed by using the polymer of Synthesis Example 2 instead of the polymer of Synthesis Example 1.

### EXAMPLE 7

A thin film transistor is manufactured in the same manner as in Example 5 except that the active layer is formed by using the polymer of Synthesis Example 3 instead of the polymer of Synthesis Example 1.

### EXAMPLE 8

A thin film transistor is manufactured in the same manner as in Example 5 except that the active layer is formed by using the polymer of Synthesis Example 4 instead of the polymer of Synthesis Example 1.

### Comparative Example 2

A thin film transistor is manufactured in the same manner as in Example 5 except that the active layer is formed by using the polymer of Comparative Synthesis Example 1 instead of the polymer of Synthesis Example 1.

### Evaluation I: Evaluation of Stretching Properties

The polymer thin films according to Examples 1 to 4 and Comparative Example 1 are respectively transferred onto a PDMS substrate and stretched by using a stretching station to evaluate stretching properties. The stretching properties are evaluated by observing crack onset strain according to elongation (0% to 50%) with an optical microscope (Leica DM4000 M LED).

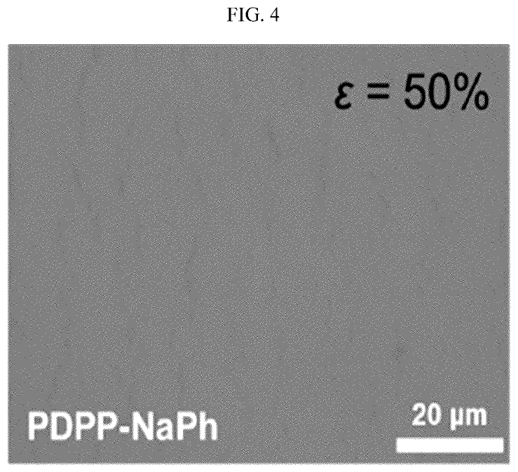

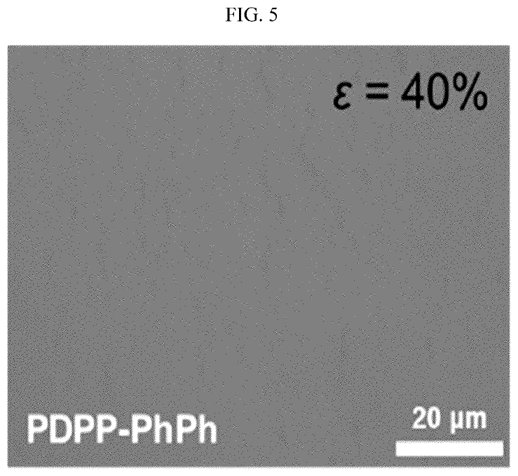

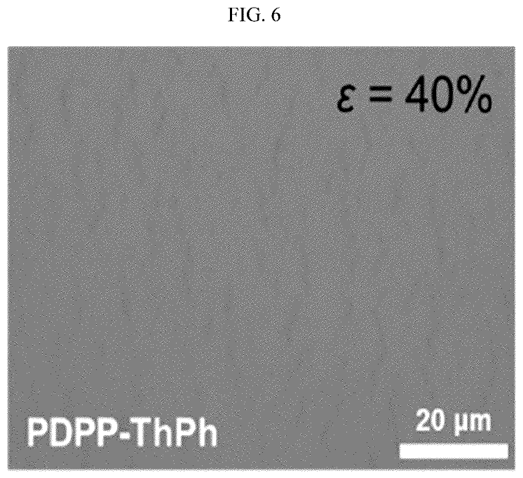

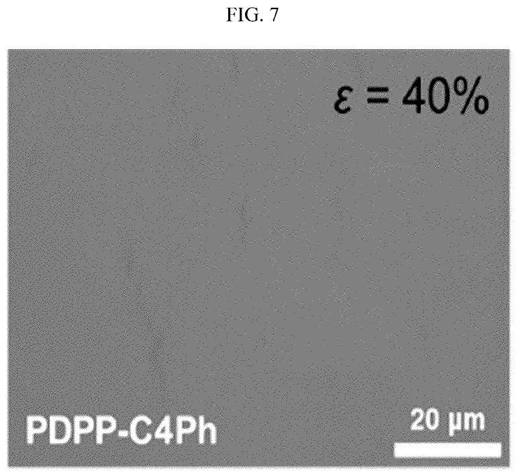

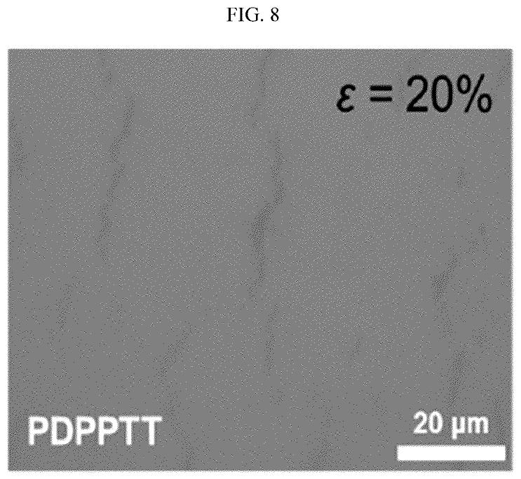

Among them, the crack evaluation results of the polymer thin films according to Examples 1, 2, 3, and 4 and Comparative Example 1 are respectively shown in FIGS. 4 to 8. FIGS. 4 to 8 are optical microscope photographs showing whether or not cracks according to elongation occur in the polymer thin films of Examples 1, 2, 3, and 4 and Comparative Example 1. Referring to FIGS. 4 to 8, the polymer thin film of Comparative Example 1 exhibits long cracks at a strain of 20%, but the polymer thin film of Example 1 exhibits cracks at a strain of 50%, and the polymer thin films of Examples 2 to 4 exhibit cracks at a strain of 40%. Accordingly, the polymer thin films of Examples 1 to 4 exhibit excellent stretching properties, compared with the polymer thin film according to Comparative Example 1.

### Evaluation II: Planarity of Compounds

Regarding Compounds 2 to 3 of the following structure constituting the polymers according to Synthesis Examples 2 to 3 and Comparative compounds 2 to 4 of the following structure, Gaussian09 SW (Method: DFT B3LYP Basis set: 6-311G (d,p)) is used to calculate a molecular skeleton of an energetically-optimized structure, and a length (X) of a long axis, the longest length, and a length (Z) of a short axis, the shortest length, of a corresponding skeleton are measured to calculate a length ratio (Z/X) of the short axis/long axis as an aspect ratio. The results are shown in Table 1.

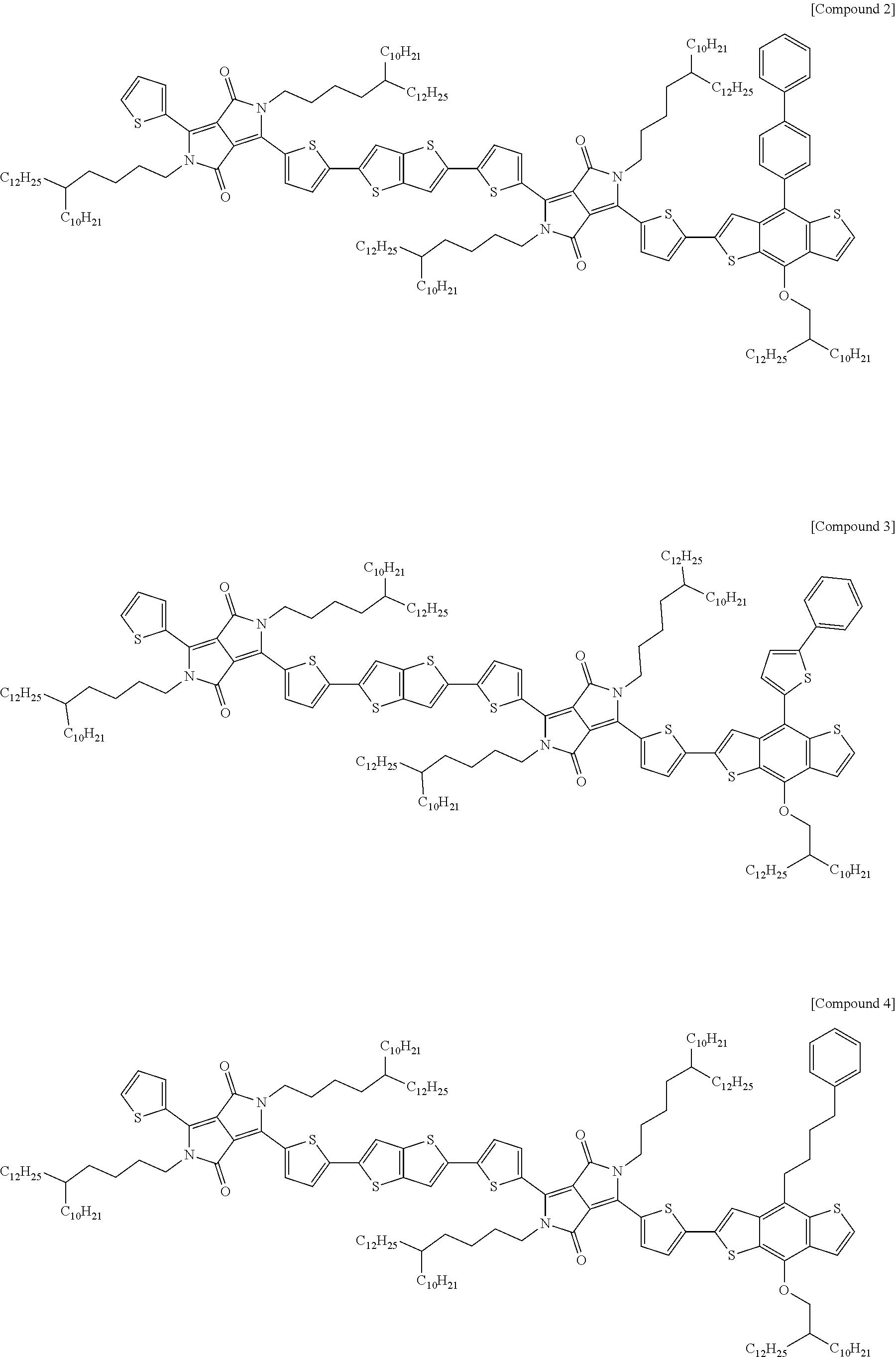

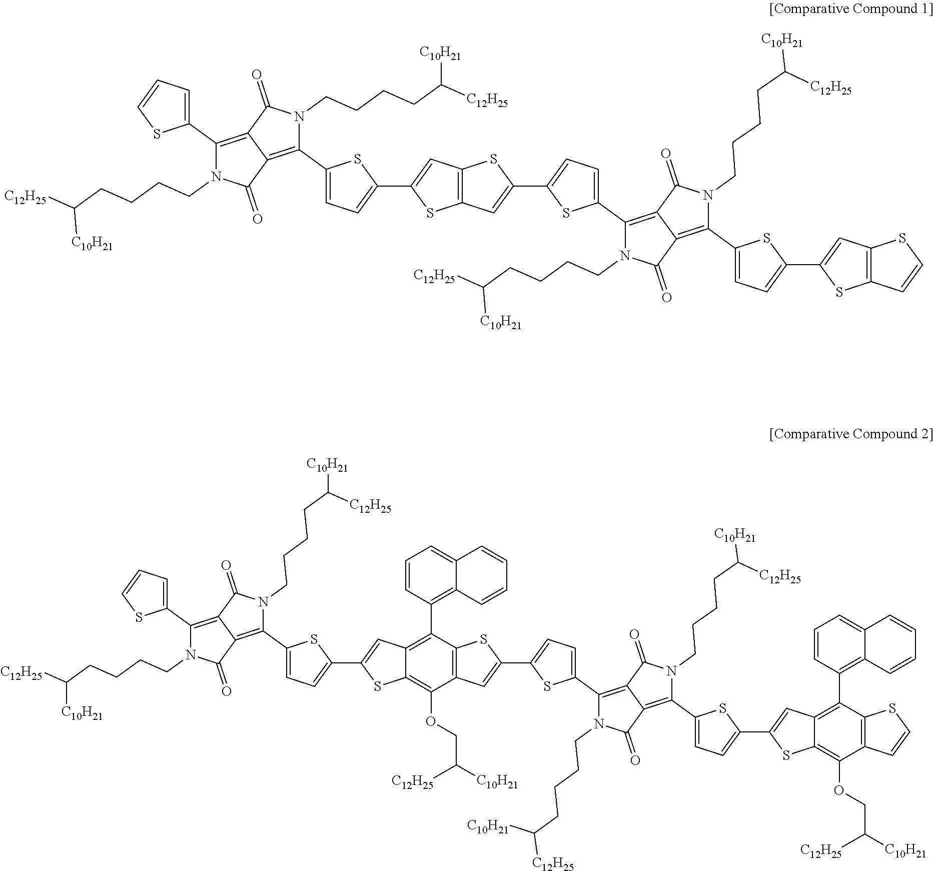

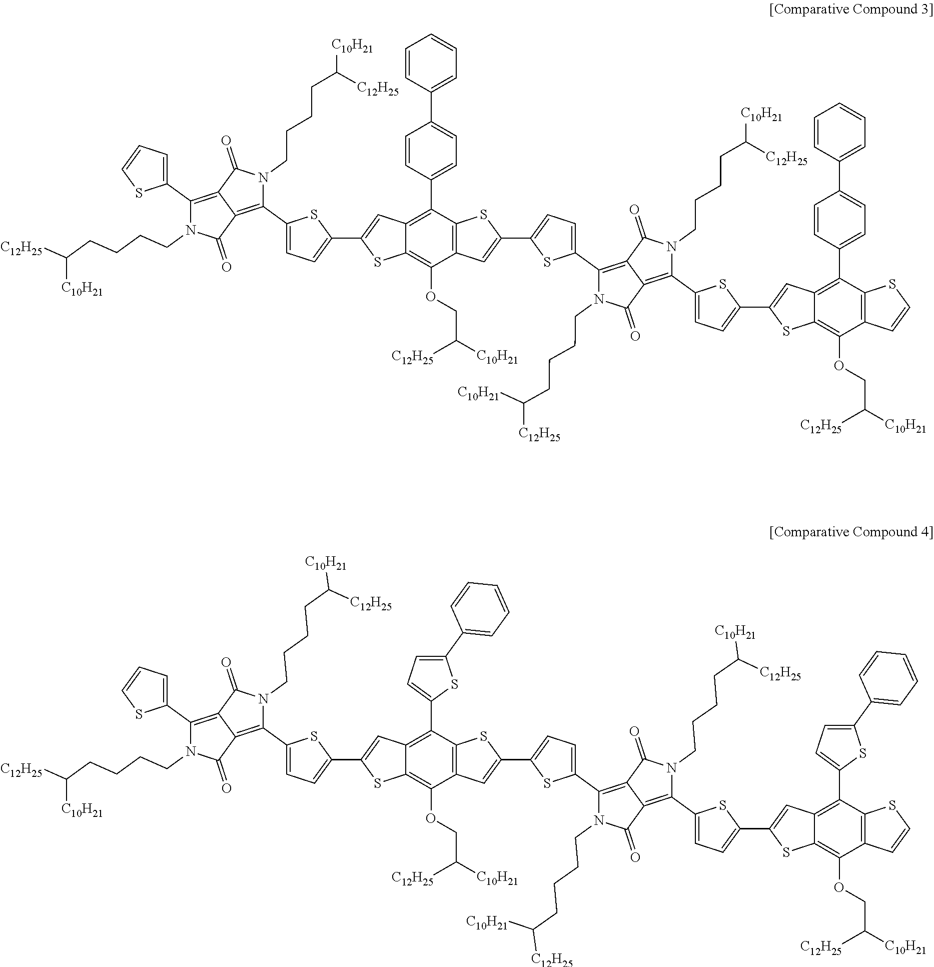

TABLE 1

| | Z-axis height (Å) | Aspect Ratio |
|---|---|---|
| Compound 2 | 4.2044 | 0.1044 |
| Compound 3 | 4.7851 | 0.1188 |
| Compound 4 | 4.0342 | 0.1001 |
| Comparative Compound 2 | 7.3581 | 0.1726 |
| Comparative Compound 3 | 5.9953 | 0.1405 |
| Comparative Compound 4 | 5.9942 | 0.1435 |

Referring to Table 1, Compounds 2 to 4 corresponding to a structure constituting a polymer according to example embodiments turn out to have excellent planarity, compared with Comparative Compound s2 to 4, wherein high planarity of a polymer is advantageous for stacking, improving charge mobility. Accordingly, the polymers containing the structures of Compounds 2 to 4 are expected to exhibit excellent charge mobility, compared with the polymers containing the structures of Comparative Compounds 2 to 4.

### Evaluation III: Charge Mobility Measurement

Charge mobility of the thin film transistors of Examples 5 to 8 and Comparative Example 2 is measured by using Semiconductor Characterization System (4200-SCS) Keithley. Among them, the results of Examples 7 and 8 and Comparative Example 2 are shown in Table 2.

TABLE 2

| | Charge mobility (cm²/Vs) |
|---|---|
| Example 7 | 0.96 |
| Example 8 | 1.0 |
| Comparative Example 2 | 0.85 |

Referring to Table 2, the thin film transistors of Examples 7 and 8 exhibit excellent charge mobility, compared with the thin film transistor of Comparative Example 2.

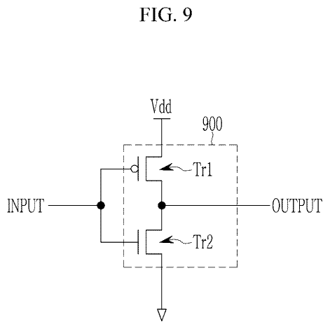

FIG. 9 is a circuit diagram of an inverter according to an example embodiment.

Referring to FIG. 9, an inverter 900 may include a PMOS transistor Tr1 and an NMOS transistor Tr2 connected between a power terminal Vdd and a ground terminal. An input node may be connected to the gates of the PMOS transistor Tr1 and the NMOS transistor Tr2. An output node may be connected to a node between the PMOS transistor Tr1 and the NMOS transistor Tr2. The inverter 900 may include any one of the thin film transistors in FIGS. 1 to 3 of the present application.

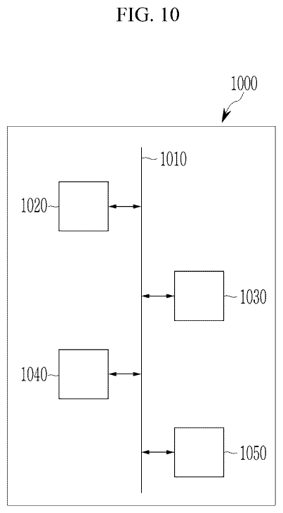

FIG. 10 is a block diagram of an electronic device according to an example embodiment.

Referring to FIG. 10, an electronic device 100 may include a processor 1020, a memory 1030, a sensor 1040, and a display 1050 that may be electrically coupled together via a bus 1010. The sensor 1040 may include an organic sensor and/or a biometric sensor and may include any one of the thin film transistors in FIGS. 1 to 3 of the present application. The memory 1030, which may be a non-transitory computer readable medium and may store a program of instructions. The memory 1030 may be a nonvolatile memory, such as a flash memory, a phase-change random access memory (PRAM), a magneto-resistive RAM (MRAM), a resistive RAM (ReRAM), or a ferro-electric RAM (FRAM), or a volatile memory, such as a static RAM (SRAM), a dynamic RAM (DRAM), or a synchronous DRAM (SDRAM). The processor 1020 may execute the stored program of instructions to perform one or more functions. For example, the processor 1020 may be configured to process electrical signals generated by the sensor 1040. The processor 1020 may be configured to generate an output (e.g., an image to be displayed on the display 1050) based on such processing. The display 1050 may include an organic light emitting device and may include any one of the thin film transistors in FIGS. 1 to 3 of the present application.

One or more of the elements disclosed above may include or be implemented in processing circuitry such as hardware including logic circuits; a hardware/software combination such as a processor executing software; or a combination thereof. For example, the processing circuitry may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc.

While this disclosure has been described in connection with what is presently considered to be practical example embodiments, it is to be understood that inventive concepts are not limited to the disclosed embodiments, but, on the contrary, are intended to cover various modifications and equivalent arrangements included within the spirit and scope of the appended claims.

What is claimed is:

1. A polymer comprising:
a first structural unit represented by Chemical Formula 1 and a second structural unit represented by Chemical Formula 2, 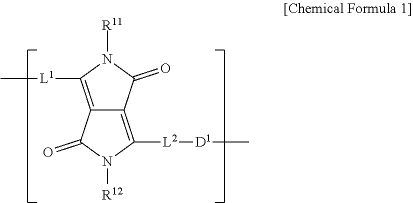
wherein, in Chemical Formula 1,
R¹¹ and R¹² are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, —COR^{a}, —OC(═O)R^{b}, —C(═O)OR^{c}, —OC(═O)OR^{d}, a halogen, a cyano group, or any combination thereof, wherein R^{a} to R^{d} are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, a cyano group, or any combination thereof,
L¹ and L² are each independently a single bond, a substituted or unsubstituted C6 to C30 arylene group, a substituted or unsubstituted divalent C3 to C30 heterocyclic group, a fused ring thereof, or any combination thereof,
D¹ is a substituted or unsubstituted C6 to C30 arylene group; a substituted or unsubstituted divalent C3 to C30 heterocyclic group including at least one of N, O, S, Se, Te, and Si; a fused ring thereof; or any combination thereof, 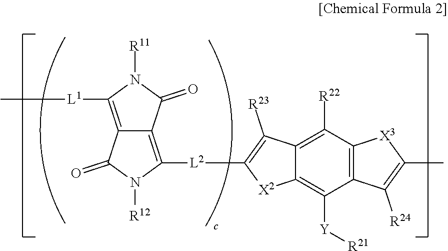
wherein, in Chemical Formula 2,
R¹¹ and R¹² are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, —COR^{a}, —OC(═O)R^{b}, —C(═O)OR^{b}, —OC(═O)OR^{d}, a halogen, a cyano group, or any combination thereof, wherein R^{a} to R^{d} are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, a cyano group, or any combination thereof,
L¹ and L² are each independently a single bond, a substituted or unsubstituted C6 to C30 arylene group, a substituted or unsubstituted divalent C3 to C30 heterocyclic group, a fused ring thereof, or any combination thereof,
c is 0 or 1,
Y is O, C(═O), —OC(═O), —C(═O)O, or —OC(═O)O, R²¹ is a substituted or unsubstituted C1 to C30 linear or branched alkyl group, a C3 to C30 linear or branched alkyl group in which at least one of non-adjacent methylene groups is replaced by oxygen (O), a substituted or unsubstituted C3 to C30 cycloalkyl group, or a substituted or unsubstituted C3 to C30 heterocycloalkyl group,
R²² is a substituted or unsubstituted C6 to C30 aryl group, a substituted or unsubstituted C7 to C30 aralkyl group, a substituted or unsubstituted C2 to C30 heteroaryl group, a substituted or unsubstituted C3 to C30 cycloalkyl group, or a substituted or unsubstituted C3 to C30 heterocycloalkyl group,
R²³ and R²⁴ are each independently hydrogen, a substituted or unsubstituted
C1 to C30 linear or branched alkyl group, a substituted or unsubstituted C1 to C30 linear or branched alkoxy group, —COR^{a}, —OC(═O)R^{b}, —C(═O)OR^{c}, —OC(═O)OR^{d}, a halogen, or a cyano group, wherein R a to R d are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a halogen, or a cyano group, and X² and X³ are each independently O, S, Se or Te.

2. The polymer of claim 1, wherein
D¹ is at least one substituted or unsubstituted phenylene group; at least one substituted or unsubstituted naphthylene group; at least one substituted or unsubstituted anthracenylene group; at least one substituted or unsubstituted phenanthrenylene group; at least one substituted or unsubstituted pentagonal ring group including at least one selected from N, O, S, Se, Te, and Si; a fused ring of two or more of the above substituted or unsubstituted pentagonal ring groups; a fused ring of at least one substituted or unsubstituted pentagonal ring group and at least one substituted or unsubstituted phenylene group; a fused ring of at least one substituted or unsubstituted pentagonal ring group and at least one substituted or unsubstituted naphthylene group; a fused ring of at least one substituted or unsubstituted pentagonal ring group and at least one substituted or unsubstituted anthracenylene group; a fused ring of at least one substituted or unsubstituted pentagonal ring group and at least one substituted or unsubstituted phenanthrenylene group; or any combination thereof.

3. The polymer of claim 1, wherein in Chemical Formula 1, D¹ is one of electron donating moieties listed in Group 1: 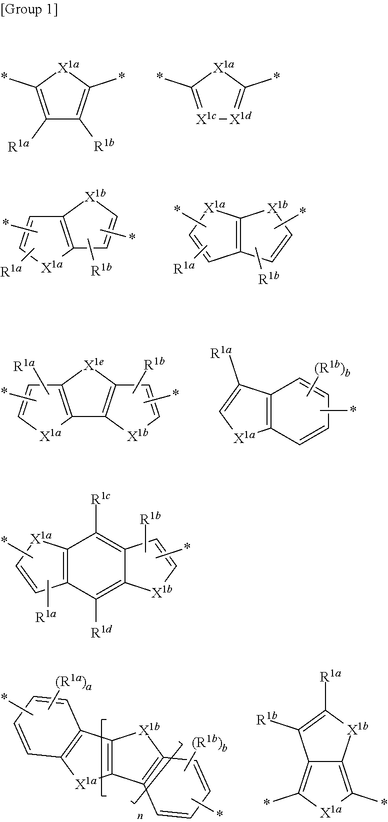
wherein, in Group 1,
X^{1a} and X^{1b} are each independently O, S, Se, or Te,
X^{1c} and X^{1d} are each independently N, CR^{x}, or SiR^{y},
X^{1e} is O, S, Se, Te, NR^{v}, CR^{w}R^{x}, or SiR^{y}R^{z},
R^{1a}, R^{1b}, R^{1c}, R^{v}, R^{w}, R^{x}, R^{y}, and R^{z} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
a and b are each independently an integer ranging from 1 to 4,
n is 0, 1, or 2, and
* is a linking point.

4. The polymer of claim 1, wherein
in Chemical Formula 1, L¹ and L² are each independently a single bond; one of electron donating moieties listed in Group 1; a substituted or unsubstituted pyridine;
a substituted or unsubstituted pyrimidine; a fused ring thereof; or any combination thereof: 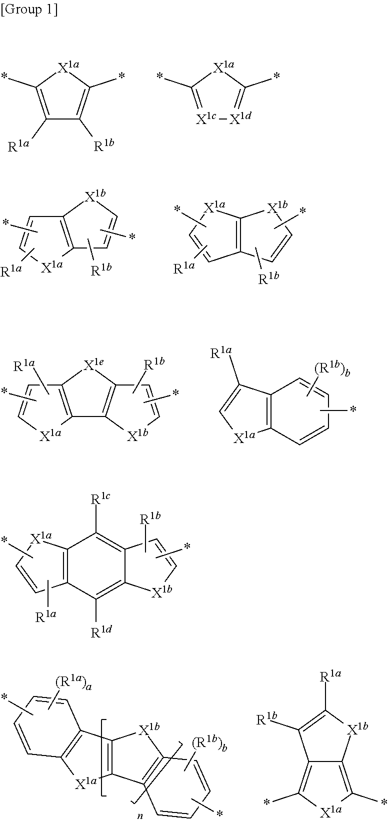
wherein, in Group 1,
X^{1a} and X^{1b} are each independently O, S, Se, or Te,
X^{1c} and X^{1d} are each independently N, CR^{x}, or SiR^{y},
X^{1e} is O, S, Se, Te, NR^{v}, CR^{w}R^{x}, or SiR^{y}R^{z},
R^{1a}, R^{1b}, R^{1c}, R^{v}, R^{w}, R^{x}, R^{y}, and R^{z} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
a and b are each independently an integer ranging from 1 to 4,
n is 0, 1, or 2, and
* is a linking point.

5. The polymer of claim 1, wherein
in Chemical Formula 1, L¹ and L² are each independently a divalent linking group including a single bond; at least one substituted or unsubstituted furan; at least one substituted or unsubstituted thiophene; at least one substituted or unsubstituted selenophene; at least one substituted or unsubstituted tellurophene; at least one substituted or unsubstituted pyrrole; at least one substituted or unsubstituted benzene;
at least one substituted or unsubstituted pyridine; at least one substituted or unsubstituted pyrimidine; or a fused ring of two or more selected from the foregoing groups; or any combination thereof.

6. The polymer of claim 1, wherein
in Chemical Formula 1, R¹¹ and R¹² are each independently a substituted or unsubstituted C6 to C30 linear alkyl group or a substituted or unsubstituted C6 to C30 branched alkyl group.

7. The polymer of claim 1, wherein
in Chemical Formula 2, R²² is one of moieties represented by Group 2: 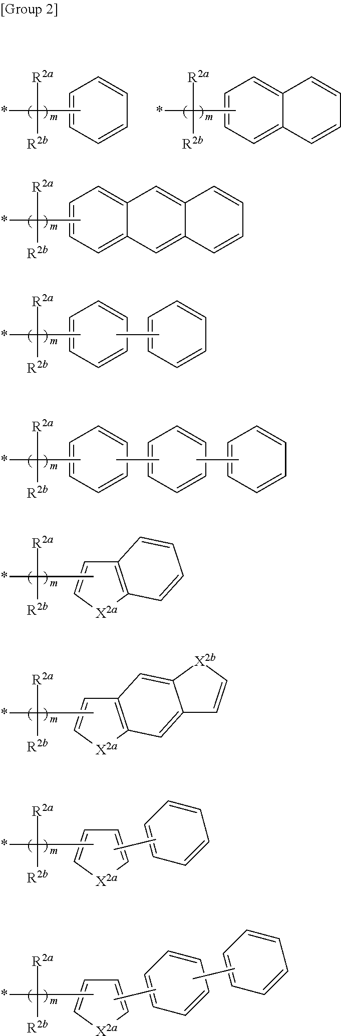
wherein, in Group 2,
X^{2a} and X^{2b} are each independently O, S, Se, or Te,
R^{2a} and R² are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
m is an integer ranging from 0 to 10,
a hydrogen atom of the benzene ring is optionally substituted or unsubstituted with a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group, and
* is a linking point.

8. The polymer of claim 7, wherein
in Group 2, at least one (CH₂) group in the benzene ring is replaced by nitrogen (N).

9. The polymer of claim 7, wherein
in —(CR^{2a}R^{2b})ₘ— of Group 2, when m is 2 or more and 10 or less, —CR^{2a}R^{2b}— that are not adjacent to each other is replaced by —O—, —S—, —C(═O)—, —OC(═O)—, —C(═O)O—, —OC(═O)O—, —S(═O)—, —S(═O)₂—, or any combination thereof.

10. The polymer of claim 1, wherein
in Chemical Formula 2, R²¹, R²³, and R²⁴ independently are a substituted or unsubstituted C6 to C30 linear alkyl group or a substituted or unsubstituted C6 to C30 branched alkyl group.

11. The polymer of claim 1, wherein
the first structural unit and the second structural unit are included in a molar ratio of about 1:9 to about 9:1.

12. The polymer of claim 1, wherein
in the polymer, an aspect ratio (Z/X) obtained by dividing a shortest axis length (Z) by a longest axis (X) length of a compound including one first structural unit and one second structural unit is less than or equal to about 1.4.

13. An organic semiconductor material comprising:
the polymer of claim 1.

14. A stretchable polymer thin film comprising:
the polymer of claim 1.

15. The stretchable polymer thin film of claim 14, further comprising:
an elastomer.

16. The stretchable polymer thin film of claim 14, wherein
when the stretchable polymer thin film is stretched by about 30%, a change in charge mobility is less than or equal to about 10%.

17. An electronic device comprising:
the stretchable polymer thin film of claim 14.

18. The electronic device of claim 17, wherein
the electronic device includes an organic diode, an organic thin film transistor, an organic solar cell, or an attachable device.
